(19) United States
(12) Patent Application Publication
(10) Pub. No.: US 2026/0144909 A1
(43) Pub. Date: May 28, 2026

(54) TETRAFLUOROETHYLENE COPOLYMER COMPOSITIONS AND ASSOCIATED METHODS

(71) Applicant: W. L. Gore & Associates, Inc., Newark, DE (US)

(72) Inventors: Valerie R. Binetti, Pennsylvania, PA (US); James L. Goepfrich, Flagstaff, AZ (US); Amanda Kamps, Newark, DE (US); David J. Minor, Phoenix, AZ (US); Brandon C. Short, Flagstaff, AZ (US); Peter D. Traylor, Flagstaff, AZ (US)

(21) Appl. No.: 19/396,964

(22) Filed: Nov. 21, 2025

Related U.S. Application Data
(60) Provisional application No. 63/724,097, filed on Nov. 22, 2024.

(51) Int. Cl. A61L 24/06; A61B 17/12; A61B 90/00; A61K 9/10; A61K 49/04; C08F 214/26

(52) U.S. Cl. CPC A61L 24/06; A61B 17/12186; A61K 9/10; A61K 49/04; A61K 49/0438; C08F 214/265; A61B 2090/3966

(57) ABSTRACT

The present disclosure is directed to a class of fluorinated copolymers, such as TFE copolymers, that can be dispersed in a liquid carrier including low toxicity liquid solvents, such as Class III Solvents, and that enable the creation of stable dispersions including the fluorinated copolymers dispersed in a liquid carrier including a water miscible organic component and an aqueous component.

### CROSS-REFERENCE TO RELATED APPLICATION

This application claims the benefit of Provisional Application No. 63/724,097, filed Nov. 22, 2024, which is incorporated herein by reference in its entirety for all purposes.

### FIELD

The present disclosure relates to fluorinated polymeric compositions, and more specifically, to tetrafluoroethylene (TFE) copolymer compositions for medical applications and associated methods of forming.

### BACKGROUND

Tetrafluoroethylene (TFE) copolymers are well known in the art. TFE copolymers are of great use in many industries, but are particularly useful in medical applications due to their inertness and biocompatibility.

While useful in many respects, utilizing TFE copolymers in liquid compositions for medical applications poses difficulties. TFE copolymers that are water soluble are not useful for many medical applications because they are not as inert or resistant to dissolution in an aqueous environment. On the other hand, TFE copolymers that are insoluble in water are often very hydrophobic, which is also problematic. In particular, the solvents used to solubilize these types of TFE copolymers can be highly toxic or otherwise detrimental to living tissue. Thus, using such a solution in vivo may not be an ideal scenario.

In addition, using TFE copolymers as embolic agents may pose additional challenges. Current embolization devices include embolic beads, glues and other liquid embolic agents. Disadvantages of these materials include lack of distal penetration, non-target embolization, permanent radiopacity and recanalization rate.

Thus, there is a need to formulate TFE copolymers in less harmful liquid carriers for use in embolization devices that have better distal penetration, more targeted embolic therapy, better radiopacity, and/or better recanalization rate.

### SUMMARY

The present disclosure is directed to fluorinated polymeric compositions, and more specifically, to tetrafluoroethylene (TFE) copolymer compositions for medical applications and associated methods of forming.

In one aspect (“Aspect 1”), a composition includes a tetrafluoroethylene copolymer dispersed in a liquid carrier; wherein a first ratio of a weight of the tetrafluoroethylene copolymer to a volume of the liquid carrier is from about 0.01 g/ml to about 0.1 g/ml.

Aspect 2 is the composition of Aspect 1, wherein the first ratio is from about 0.02 g/ml to about 0.08 g/ml.

Aspect 3 is the composition of Aspect 2, wherein the first ratio is from about 0.03 g/ml to about 0.06 g/ml.

Aspect 4 is the composition of any one of Aspects 1-3, further including: a radiopacity agent suspended in the liquid carrier; wherein a second ratio of a weight of the radiopacity agent to the volume of the liquid carrier is from about 0.1 g/ml to about 0.75 g/ml.

Aspect 5 is the composition of Aspect 4, wherein the second ratio is from about 0.12 g/ml to about 0.5 g/ml.

Aspect 6 is the composition of Aspect 5, wherein the second ratio is from about 0.3 g/ml to about 0.4 g/ml.

Aspect 7 is the composition of any of the preceding Aspects, wherein the liquid carrier includes a water miscible organic component and an aqueous component; wherein the ratio of the water miscible organic component to the aqueous component is from about 1:1 to about 4:1.

Aspect 8 is the composition of Aspect 7, wherein the ratio of the water miscible organic component to the aqueous component is from about 3:2 to about 7:3.

Aspect 9 is the composition of Aspect 7, wherein the water miscible organic component is selected from a group consisting of alcohols, esters, ketones, glycols, and aldehydes, and polar aprotic solvents.

Aspect 10 is the composition of Aspect 7, wherein the water miscible organic component is selected from the group consisting of FDA Class III Solvents.

Aspect 11 is the composition of Aspect 7, wherein the water miscible organic component includes propylene glycol (PG), ethanol, or dimethyl sulfoxide (DMSO).

Aspect 12 is the composition of Aspect 7, wherein the water miscible organic component is configured to stabilize the tetrafluoroethylene copolymer dispersed in the liquid carrier by forming physical crosslinks with the tetrafluoroethylene copolymer.

Aspect 13 is the composition of Aspect 12, wherein the physical crosslinks include hydrogen bonds.

Aspect 14 is the composition of Aspect 7, wherein the aqueous component includes water or saline including phosphate buffered saline (PBS) or saline.

Aspect 15 is the composition of any of Aspects 4 to 14, wherein the composition is visible within a patient's body under fluoroscopy with wavelength of from about 0.01 to about 10 nm.

Aspect 16 is the composition of any one of Aspects 4 to 15, wherein the radiopacity agent is selected from a group consisting of tantalum, barium sulfate, iohexol, iodixanol, ioxaglic acid, lipiodol, and triiodobenzoic acid.

Aspect 17 is the composition of any of the preceding Aspects, wherein the tetrafluoroethylene copolymer includes a hydroxyl functional group.

Aspect 18 is the composition of any of the preceding Aspects, wherein the tetrafluoroethylene copolymer is poly(tetrafluoroethylene-co-vinyl alcohol) (TFE-VOH).

Aspect 19 is the composition of any of the preceding Aspects, wherein the tetrafluoroethylene copolymer has a molecular weight of from about 50,000 g/mol to about 500,000 g/mol.

Aspect 20 is the composition of any of the preceding Aspects, wherein the tetrafluoroethylene copolymer has a molecular size of from about 33 nm to about 165 nm.

Aspect 21 is the composition of any of the preceding Aspects, wherein the composition is configured to be injected into a target space to form a gel that occludes at least a portion of the space.

Aspect 22 is the composition of Aspect 21, wherein the target space is within a vessel, an organ, or a tissue of a patient.

Aspect 23 is the composition of Aspect 21 or 22, wherein the composition is injected using a delivery device including a needle, a catheter, a microcatheter, or a syringe.

Aspect 24 is the composition of any one of Aspects 21 to 23, wherein the target space is adjacent to a distal end of the delivery device.

Aspect 25 is the composition of any one of Aspects 21 to 23, wherein the target space is downstream and a distance away from the distal end of the delivery device, wherein the composition is conveyed to the target site by the fluid flowing through the vessel.

Aspect 26 is the composition of any one of Aspects 21 to 25, wherein the composition is an embolic agent used for an embolization procedure.

Aspect 27 is the composition of any one of Aspects 21 to 26, wherein the composition is stirred or agitated before injection.

Aspect 28 is the composition of any one of Aspects 21 to 27, wherein the tetrafluoroethylene copolymer and the radiopacity agent are evenly distributed within the gel formed at the target space.

Aspect 29 is the composition of any of the preceding Aspects, wherein the tetrafluoroethylene copolymer dispersed in the liquid carrier forms a dispersion kinetically stable at 25° C. for at least 5 minutes without stirring or agitation.

Aspect 30 is the composition of any of the preceding Aspects, wherein the dispersion is kinetically stable at 25° C. for at least 4 months without stirring or agitation.

Aspect 31 is the composition of any of the preceding Aspects, wherein the dispersion is kinetically stable at 25° C. for at least 2 years without stirring or agitation.

In another aspect (“Aspect 32”), a method of preparing a composition includes: mixing a tetrafluoroethylene copolymer with a water miscible organic component to form a mixture; mixing an aqueous component with the mixture to form a kinetically stable dispersion; wherein the composition includes the tetrafluoroethylene copolymer uniformly distributed throughout a liquid carrier including the water miscible organic component and the aqueous component.

Aspect 33 is the method of Aspect 32, further including adding a radiopacity agent to the kinetically stable dispersion to form the composition, and wherein the radiopacity agent is suspended in the liquid carrier.

In another aspect (“Aspect 34”), a method of preparing a composition includes: mixing a tetrafluoroethylene copolymer with a liquid carrier including a water miscible organic component and an aqueous component to form a kinetically stable dispersion; wherein the composition includes the tetrafluoroethylene copolymer uniformly distributed throughout a liquid carrier including the water miscible organic component and the aqueous component.

Aspect 35 is the method of Aspect 34, further including adding a radiopacity agent to the kinetically stable dispersion to form the composition, and wherein the radiopacity agent is suspended in the liquid carrier.

Aspect 36 is the method of Aspects 32 or 34, wherein the mixing steps are conducted using a tumbling fixture at a temperature of from about 65° C. to about 105° C.

Aspect 37 is the method of Aspects 32 or 34, wherein the mixing steps are conducted using a shaker plate at a temperature of from about 60° C. to about 100° C.

Aspect 38 is the method of Aspects 32 or 34, wherein the mixing steps are conducted via stirring at a temperature of from about 60° C. to about 100° C.

Aspect 39 is the method of any one of Aspects 32 to 38, wherein the radiopacity includes tantalum.

In another aspect (“Aspect 40”), a method of preparing a composition includes: mixing a tetrafluoroethylene copolymer with a water miscible organic component to form a mixture; mixing an aqueous component with the mixture to form a kinetically stable dispersion; wherein the composition includes the tetrafluoroethylene copolymer uniformly distributed throughout a liquid carrier including the water miscible organic component and the aqueous component.

Aspect 41 is the method of Aspect 40, further including mixing a radiopacity agent with the kinetically stable dispersion to form the composition, and wherein the radiopacity agent is uniformly distributed throughout a liquid carrier including the water miscible organic component and the aqueous component.

Aspect 42 is the method of Aspects 40 or 41, wherein the first and second mixing steps are conducted using a tumbling fixture at a temperature of from about 65° C. to about 105° C.

Aspect 43 is the method of Aspects 40 or 41, wherein the first and second mixing steps are conducted using a shaker plate at a temperature of from about 60° C. to about 100° C.

Aspect 44 is the method of Aspects 40 or 41, wherein the first and second mixing steps are conducted via stirring at a temperature of from about 60° C. to about 100° C.

Aspect 45 is the method of any one of Aspects 40 to 44, wherein the third mixing step is conducted at a temperature of from about 30° C. to about 70° C.

In another aspect (“Aspect 46”), a method of preparing a composition includes: mixing a tetrafluoroethylene copolymer with a liquid carrier including a water miscible organic component and an aqueous component to form a kinetically stable dispersion; wherein the composition includes the tetrafluoroethylene copolymer uniformly distributed throughout a liquid carrier including the water miscible organic component and the aqueous component.

Aspect 47 is the method of Aspect 46, further including adding a radiopacity agent to the kinetically stable dispersion to form the composition, and wherein the radiopacity agent is suspended in the liquid carrier.

Aspect 48 is the method of Aspects 46 or 47, wherein the first mixing step is conducted using a tumbling fixture at a temperature of from about 65° C. to about 105° C.

Aspect 49 is the method of Aspects 46 or 47, wherein the first mixing step is conducted using a shaker plate at a temperature of from about 60° C. to about 100° C.

Aspect 50 is the method of Aspects 46 or 47, wherein the first mixing step is conducted via stirring at a temperature of from about 60° C. to about 100° C.

Aspect 51 is the method of any one of Aspects 46 to 50, wherein the second mixing step is conducted at a temperature of from about 30° C. to about 70° C.

Aspect 52 is the method of any one of Aspects 40 to 51, wherein the radiopacity agent includes iohexol.

In another aspect (“Aspect 53”), a method includes: providing a composition including a tetrafluoroethylene copolymer uniformly distributed throughout a liquid carrier, the liquid carrier including a water miscible organic component and an aqueous component; introducing the composition into a patient at a target space; and diffusing the water miscible organic component into the patient to form a gel including the tetrafluoroethylene copolymer at the target space.

Aspect 54 is the method of Aspect 53, further including a radiopacity agent suspended in the liquid carrier.

Aspect 55 is the method of Aspects 53 or 54, wherein the composition is introduced into an occlusion site or a bulking site of the patient.

Aspect 56 is the method of Aspect 55, wherein the occlusion site or the bulking site is within a vessel, an organ, or a tissue of the patient.

Aspect 57 is the method of any one of Aspects 53 to 56, wherein the introducing step is performed using a delivery device including a needle, a catheter, a microcatheter, or a syringe.

Aspect 58 is the method of any one of Aspects 53 to 57, wherein the gel is substantially porous.

Aspect 59 is the method of any one of Aspects 53 to 58, wherein the gel is permeable.

Aspect 60 is the method of any one of Aspects 53 to 59, wherein the composition is an embolic agent used for an embolization procedure.

Aspect 61 is the method of any one of Aspects 53 to 60, wherein the radiopacity agent includes tantalum; wherein the composition is stirred or agitated before the introducing step.

Aspect 62 is the composition of any one of Aspects 53 to 61, wherein the tetrafluoroethylene copolymer and the radiopacity agent are evenly distributed within the gel formed at the target space.

In another Aspect (“Aspect 63”), a method of treating disease by providing distal embolization includes: providing a composition including a tetrafluoroethylene copolymer uniformly distributed throughout a liquid carrier, the liquid carrier including a water miscible organic component and an aqueous component; and deploying the composition through a delivery system operable to deliver the composition to a target site.

Aspect 64 is the method of Aspect 63, wherein deploying the composition through a delivery system includes preparing a microcatheter by priming with a primer solution; and deploying the composition through the microcatheter operable to deliver the composition to a target site in peripheral arterial vasculature; and achieving occlusion of the target site.

Aspect 65 is the method of Aspect 63, wherein deploying the composition through the microcatheter operable to deliver the composition to a target site further includes deploying the composition through the microcatheter operable to deliver the composition to a target site adjacent to the microcatheter distal end.

Aspect 66 is the method of Aspect 63, wherein deploying the composition through the microcatheter operable to deliver the composition to a target site further includes deploying the composition through the microcatheter operable to deliver the composition to a target site downstream and a distance way from the microcatheter distal end, wherein the composition is conveyed by the flow of blood through the vessel.

Aspect 67 is the method of Aspect 63, wherein deploying the composition further includes deploying the composition through the microcatheter operable to deliver the composition to a target site so as to achieve occlusion of the target site.

Aspect 68 is the method of Aspect 63, wherein deploying the composition further includes deploying the composition through the microcatheter operable to deliver the composition to a target site so as to achieve occlusion of the target site in peripheral arterial vasculature.

Aspect 69 is the method of any one of Aspects 63 to 68, wherein the distal embolization is for treatment of hemorrhage.

Aspect 70 is the method of any one of Aspects 63 to 68, wherein the distal embolization is for treatment of hypervascular tumors.

Aspect 71 is the method of any one of Aspects 63 to 68, wherein the distal embolization is for treatment of pseudoaneurysms.

Aspect 72 is the method of any one of Aspects 63 to 68, wherein the distal embolization is for treatment of aneurysms.

Aspect 73 is the method of any one of Aspects 63 to 68, wherein the distal embolization is for renal embolization.

Aspect 74 is the method of any one of Aspects 63 to 68, wherein the distal embolization is for neurovascular indications.

Aspect 75 is the method of any one of Aspects 69 to 73, wherein the distal embolization is performed within or involves the portal vein.

### BRIEF DESCRIPTION OF THE DRAWINGS

The accompanying drawings are included to provide a further understanding of the disclosure, and are incorporated in and constitute a part of this specification, illustrate embodiments of the disclosure, and together with the description, serve to explain the principles of the disclosure.

FIG. 1 is a schematic diagram of a composition including a tetrafluoroethylene (TFE) copolymer dispersed therein, according to certain embodiments of the present disclosure.

FIGS. 2A and 2B are schematic diagrams of an exemplary device configured to deliver a composition including a tetrafluoroethylene (TFE) copolymer dispersed therein, according to certain embodiments of the present disclosure.

FIG. 3 is a flow diagram illustrating a process of making a composition including a tetrafluoroethylene (TFE) copolymer dispersed therein, according to certain embodiments of the present disclosure.

FIGS. 4A-4D are TEM images of a composition including a tetrafluoroethylene (TFE) copolymer, according to certain embodiments of the present disclosure.

FIG. 5 shows DLS data of samples described in Example 2, according to certain embodiments of the present disclosure.

FIGS. 6A-6B are fluoroscopy images of various compositions including a tetrafluoroethylene (TFE) copolymer and a radiopacity agent, according to certain embodiments of the present disclosure.

FIGS. 7A-7C are fluoroscopy images of various stages of an embolization treatment procedure as described in Example 20, according to certain embodiments of the present disclosure.

Persons skilled in the art will readily appreciate that various aspects of the present disclosure can be realized by any number of methods and apparatuses capable of performing the intended functions. Stated differently, other methods and apparatuses can be incorporated herein to perform the intended functions. It should also be noted that the accompanying drawing figures referred to herein are not all drawn to scale, but can be exaggerated to illustrate various aspects of the present disclosure, and in that regard, the drawing figures should not be construed as limiting.

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

As used herein, a “dispersion” is a homogeneous mixture having a uniform distribution of solid particles throughout a continuous medium (e.g., a liquid carrier). The size of the solid particles may range from about 0.001 micrometers to about 1 micrometer (i.e., 1 nanometer to 1000 nanometers). Since a dispersion is a homogeneous mixture, the solid particles distributed throughout a dispersion do not tend to settle down or separate from the medium if the dispersion is left undisturbed.

As used herein, a “suspension” is a heterogeneous mixture having a non-uniform distribution of solid particles throughout a continuous medium (e.g., a liquid carrier). The size of the solid particles may range from about 0.5 micrometers to about 9 micrometers (i.e., 500 nanometers to 9000 nanometers). Unlike a dispersion, the solid particles distributed throughout a suspension are sufficiently large for sedimentation, and tends to separate from the medium if the suspension is left undisturbed.

As used herein, an “emulsion” is a homogeneous mixture having a uniform distribution of a discontinuous first liquid throughout a continuous second liquid, the first liquid and second liquid being immiscible with each other.

As used herein, a “colloid” is a first substance microscopically or nanoscopically distributed evenly throughout a second substance. A “colloid” may be used to refer to a “dispersion” when the first substance is a solid and the second substance is a liquid. A “colloid” may also be used to refer to an “emulsion” when the first substance is a liquid and the second substance is another liquid.

As used herein, a “solution” is a homogeneous mixture having one or more solutes dissolved in a solvent. The solutes may include solid particles having particles sizes smaller than 0.001 micrometers (i.e., 1 nm) when dissolved in a liquid solvent. Alternatively or additionally, the solutes may include a first liquid miscible with the solvent (e.g., a second liquid).

As used herein, “water miscible” means the property of a component to mix in all proportions with water, to form a homogeneous solution.

As used herein, a “solvent” is any substance that dissolves a solute, resulting in a solution. In some embodiments as disclosed herein, a solvent may form a solution which is then used as a liquid carrier for another substance that does not dissolve in the solvent or solution. For example, a composition may include solid particles dispersed or suspended, but not dissolved, in a solution, the solution having an aqueous component (i.e., a solute) dissolved in a water miscible organic component (i.e., a solvent).

As used herein, “low toxicity solvents” include “Class III Solvents” as defined by the U.S. Food and Drug Administration (FDA) or the International Conference on Harmonixation of Technical Requirements for Registration of Pharmaceuticals for Human Use (ICH) such as acetone and dimethyl sulfoxide and solvents “Generally Recognized as Safe” (GRAS) as defined by the FDA such as propylene glycol (See Guidance for Industry: Q3C: Tables and List”, FDA Guidance, Rev 1, November 2003 and “ICH Guideline: Harmonization of Residual Solvents in Pharmaceuticals”, L S Wigman, Pharm Tech, p 102-108, October 1996.) In addition, a solvent can also include any water miscible solvent with relatively low toxicity, e.g., some that are FDA and/or ICH classified Class 2 solvents with water miscibility and relatively low toxicity, such as acetonitrile, dioxane, formamide, dimethylformamide, pyridine, NMP, methylpyrrolidone, dimethylacetamide, ethylene glycol, methyoxymethanol, pyridine, piperidine, sulfolane, tetrahydrofuran, trichloroacetic acid, and the like. Other suitable low toxicity solvents include oligoethylene glycol such as polyethylene glycol and polyethylene oxide. Table 1 (adopted from the FDA Guidance document “Class III Solvents”) includes a list of Class III Solvents.

TABLE 1
FDA Class III Solvents

| | |
|---|---|
| Acetic Acid | Isopropyl acetate |
| Acetone | Methyl acetate |
| 1-Butanol | 3-Methyl-1-butanol |
| 2-Butanol | Methylethyl ketone |
| Butyl acetate | Methylisobutyl |
| | ketone |
| Dimethyl | 2-Methyl-1-propanol |
| sulfoxide | |
| Ethanol | 1-Pentanol |
| Ethyl acetate | 1-Propanol |
| Ethyl formate | 2-Propanol |
| Formic Acid | Propyl acetate |
| Isobutyl acetate | Methyl acetate |

As used herein, “kinetically stable” is a degree of stability wherein a component of a homogeneous mixture remains uniformly distributed (i.e., precipitation, flocculation, sedimentation, separation, or coalescence of a component is not visibly detectable with the naked eye) for at least one minute, at least 5 minutes, at least 30 minutes, at least 60 minutes, at least one month, at least 4 months, or at least one year.

As used herein, a “gel” is a material composed of long chains of polymers that are cross-linked to form a three-dimensional network structure, capable of absorbing and retaining large amounts of water or other liquids.

As used herein, an “occlusion site” or a “bulking site” are used to collectively refer to a tissue, an empty space, gap, or defect surrounded by tissue, a lumen of a vessel, or a complex system of vessels.

As used herein, “TFE-VOH” refers to poly(tetrafluoroethylene-co-vinyl alcohol).

As used herein, “PG” refers to propylene glycol.

As used herein, “PBS” refers to phosphate buffered saline.

As used herein, “DMSO” refers to dimethyl sulfoxide.

### II. Overview

Generally, fluoropolymers fall into one of two categories: those that dissolve in non-aqueous and often toxic solvents and those that are highly water-soluble. For the non-water soluble fluoropolymers, the use of combining surfactants and fluoropolymers to form emulsions is well known. These admixtures in the form of emulsions allow for either hydrophobic or hydrophilic agents to be admixed with fluoropolymers. However, fluoropolymers having surfactant-like properties, i.e., that have the ability to form a kinetically stable composition in the presence of a hydrophilic agent, are not known in the art. The ability of the fluorinated co-polymers of the disclosure to form kinetically stable compositions for medical applications without the addition of another component, such as a surfactant, is unique.

The present disclosure is directed to compositions that include fluorinated copolymers, such as TFE copolymers, that are dispersed in a liquid carrier that includes a water miscible organic component and an aqueous component to form a kinetically stable dispersion. In certain embodiments, the water miscible organic component may be selected from the list of FDA Class Ill Solvents listed in Table 1. In some embodiments, the water miscible organic component may include low toxicity solvents including, for example, propylene glycol (PG). In certain embodiments, the aqueous component may be selected from water and/or saline (e.g., phosphate buffered saline (PBS) and/or saline and/or 0.9% USP Sterlie Saline). In some embodiments, the TFE copolymers may include copolymers of TFE with functional monomers that include acetate, alcohol, amine or amide functional groups, as well as combinations thereof, such as poly(tetrafluoroethylene-vinyl alcohol) (TFE-VOH). The fluorinated copolymers of the current disclosure can form kinetically stable dispersions in the liquid carrier in the absence of an additional surfactant.

According to some embodiments, the composition may further include a radiopacity agent including a material that does not allow radiation (e.g., X-ray, fluoroscopy) to pass through it, for example tantalum, barium sulfate, iohexol, iodixanol, ioxaglic acid, lipiodol, and triiodobenzoic acid, iopamidol iopromide, and/or gold nanoparticles. A radiopacity agent may be included in the composition to provide visibility during the introduction of such composition into a patient's body under fluoroscopy of a certain wavelength. In some embodiments, the composition includes a radiopacity agent dispersed in the liquid carrier, forming a kinetically stable dispersion of the TFE copolymer and the radiopacity agent. In certain embodiments, the composition includes a radiopacity agent suspended in the liquid carrier, forming a mixture of a kinetically stable dispersion of the TFE copolymer with a suspension of the radiopacity agent in the liquid carrier.

Other embodiments described herein include methods of making and using said compositions, along with devices, systems, and methods utilizing additional devices utilized in the delivery or application of the described compositions.

### III. Compositions

FIG. 1 is a schematic diagram of a composition including a tetrafluoroethylene (TFE) copolymer dispersed therein, according to certain embodiments of the present disclosure. As shown, the composition 100 includes a water miscible organic component 102, an aqueous component 104, and a copolymer 106, the water miscible organic component 102 and the aqueous component 104 collectively forming a homogeneous solution acting as a liquid carrier for the copolymer 106 and/or the radiopacity agent 108. In certain embodiments, the composition 100 is a kinetically stable dispersion including the copolymer 106 dispersed in the liquid carrier including the water miscible organic component 102 and the aqueous component 104. In some embodiments, the composition 100 is a kinetically stable dispersion including the copolymer 106 dispersed in the liquid carrier without use of a surfactant. In some embodiments, the composition 100 is a kinetically stable dispersion including the copolymer 106 dispersed in the liquid carrier without use of a co-solvent. In certain embodiments, the composition 100 may further include a radiopacity agent 108 dispersed or suspended in the liquid carrier.

The water miscible organic component 102 may include alcohols, esters, ketones, glycols (e.g., propylene glycol, alkylene glycol, aqueous alkylene glycol, oligoethylene glycol, oligopropylene glycol), and aldehydes, and polar aprotic solvents (e.g., DMSO, NMP). Propylene glycol, oligoethylene glycol, and their aqueous cosolvents, in particular, are known for their extremely low toxicity, widespread intravenous pharmaceutical utility, and are classified as Generally Recognized as Safe (GRAS). In certain embodiments, the water miscible organic component 102 include a low toxicity solvent. In some embodiments, the water miscible organic component 102 may be selected from the group consisting of FDA Class III Solvents as listed in Table 1. In certain embodiments, the water miscible organic component 102 includes propylene glycol (PG), ethanol, and/or dimethyl sulfoxide (DMSO).

The aqueous component 104 may include water or saline. In some embodiments, the aqueous component 104 includes phosphate buffered saline (PBS), saline, or 0.9% USP Sterile Saline. Since the organic component 102 is water miscible, the aqueous component 104 dissolves in the organic component 102 when mixed together with the organic component to form a solution that is configured to act as a liquid carrier for particles (e.g., copolymer 106 and/or radiopacity agent 108).

In some embodiments, the pre-mixing volume ratio of the water miscible organic component 102 to the aqueous component 104 is from about 1:1 to about 4:1, or from about 3:2 to about 4:1, or from about 2:1 to about 4:1, or from about 5:2 to about 4:1, or from about 3:1 to about 4:1, or from about 1:1 to 11:3, or from about 3:2 to about 11:3, or from about 2:1 to about 11:3, or from about 5:2 to about 11:3, or from about 3:1 to about 11:3, or from about 1:1 to 11:3, or from about 3:2 to about 10:3, or from about 2:1 to about 10:3, or from about 5:2 to about 10:3, or from about 3:1 to about 10:3, or from about 1:1 to 3:1, or from about 3:2 to about 3:1, or from about 2:1 to about 3:1, or from about 5:2 to about 3:1, or from about 1:1 to 8:3, or from about 3:2 to about 8:3, or from about 2:1 to about 8:3, or from about 5:2 to about 8:3, or from about 1:1 to 7:3, or from about 3:2 to about 7:3, or from about 2:1 to about 7:3, or within any range encompassing any two of these values as endpoints. In some embodiments, the composition 100 includes a water miscible organic component 102 mixed with at least about 50%, about 47%, about 45%, about 40%, about 35%, about 30%, about 27%, about 25%, about 23%, about 22% of the aqueous component 104 compared to the total volume of the liquid carrier, or within any range encompassing any two of these values as endpoints.

The copolymer 106 may be selected from a class of fluorinated copolymers that can be dispersed in the water miscible organic component 102 upon mixing, thus forming a kinetically stable dispersion of the copolymer 106 dispersed in a liquid carrier including the water miscible organic component 102. The formed kinetically stable dispersion is a homogeneous mixture having a uniform distribution of particles of the copolymer 106 throughout a continuous medium (e.g., the liquid carrier including the organic component 102). Since a dispersion is a homogeneous mixture, the solid particles distributed throughout a dispersion do not tend to settle down or separate from the medium if the dispersion is left undisturbed. The composition 100 including the formed dispersion may be kinetically stable at 25° C. for at least 5 minutes, at least 10 minutes, at least 15 minutes, at least 30 minutes, at least 1 hour, at least 2 hours, at least 5 hours, at least 10 hours, at least 24 hours, at least 2 days, at least 15 days, at least 30 day, at least 2 months, at least 4 months, at least 6 months, at least 8 months, at least 12 months, at least 15 months, at least 18 months, at least 21 months, or at least 3 years without stirring or agitation.

As illustrated in FIG. 1, and without wishing to be bound by any particular theory, the water miscible organic component 102 is configured to stabilize the copolymer 106 dispersed in the liquid carrier by forming physical crosslinks 110 with the copolymer 106, thus forming a kinetically stable composition with particles of the copolymer 106 uniformly distributed throughout the liquid carrier including the organic component 102. In some embodiments, the physical crosslinks 110 include hydrogen bonds. The composition 100 may be formed and transferred to a vial 112 that is subsequently sealed, and may be stored at room temperature with the dispersion staying kinetically stable for at least 5 minutes, at least 10 minutes, at least 15 minutes, at least 30 minutes, at least 1 hour, at least 2 hours, at least 5 hours, at least 10 hours, at least 24 hours, at least 2 days, at least 15 days, at least 30 day, at least 2 months, at least 4 months, at least 6 months, at least 8 months, at least 12 months, at least 15 months, at least 18 months, at least 21 months, or at least 2 years without stirring or agitation.

The copolymer 106 may include TFE copolymers that include at least about 77 mole % of organic functional groups including acetate, alcohol (i.e., hydroxyl), amine, or amide, or combinations thereof, and at least about 15 mole % of TFE. Methods of making fluorinated copolymers of the disclosure are described below and are known in the art (see e.g., Modena et al., “Vinyl Acetate and Vinyl Alcohol Copolymers with Tetrafluoroethylene,” European Polymer Journal, 1967, v. 3, pp. 5-12).

In some embodiments, the copolymer 106 is a TFE copolymer including, for example, poly(tetrafluoroethylene-co-vinyl alcohol) (TFE-VOH). In some embodiments, the copolymer 106 is TFE-VOH. In certain embodiments, the copolymer 106 is [TFE]ₙ-[VOH]ₘ, wherein the monomer ratio “n” of TFE is in a range of from about 15 to about 24, or from about 15.5 to about 23.5, or from about 16 to about 23, or from about 16.5 to about 22.5, or from about 17 to about 22, or from about 17.5 to about 21.5, or from about 18 to about 21, or from about 18.5 to about 21, or from about 19 to about 21, or from about 19.5 to about 20.5, or within any range encompassing any two of these values as endpoints, and wherein the monomer ratio “m” of VOH is in a range of from about 76 to about 85, or from about 76.5 to about 84.5, or from about 77 to about 84, or from about 77.5 to about 83.5, or from about 78 to about 83, or from about 78.5 to about 82.5, or from about 79 to about 82, or from about 79 to about 81.5, or from about 79 to about 81, or from about 79.5 to about 80.5, or within any range encompassing any two of these values as endpoints. TFE-VOH copolymers with a TFE mole content as low as 15.5% are typically insoluble in water.

As disclosed in embodiments herein and further shown in the Examples below, the copolymer 106 includes a TFE-VOH copolymer with a TFE mole content as low as 15.5%. The copolymer 106 is dispersed in a liquid carrier having a water miscible organic component mixed with 20% (v/v) to 50% (v/v) of an aqueous component (e.g., water and/or saline), thus forming a composition 100 that is a kinetically stable dispersion at physiological temperatures. The copolymer 106 self assembles upon contact of the composition 100 with blood or other bodily fluids and hardens to form solid or gel-like embolic masses, as a congealed mass, or as multiple, discrete particles.

In certain embodiments, the copolymer 106 is a TFE copolymer having a molecular weight of from about 50,000 g/mol to about 500,000 g/mol, or from about 75,000 g/mol to about 500,000 g/mol, or from about 100,000 g/mol to about 500,000 g/mol, or from about 125,000 g/mol to about 500,000 g/mol, or from about 150,000 g/mol to about 500,000 g/mol, or from about 175,000 g/mol to about 500,000 g/mol, or from about 200,000 g/mol to about 500,000 g/mol, or from about 200,000 g/mol to about 450,000 g/mol, or from about 200,000 g/mol to about 400,000 g/mol, or from about 200,000 g/mol to about 350,000 g/mol, or from about 200,000 g/mol to about 300,000 g/mol, or from about 200,000 g/mol to about 250,000 g/mol, or within any range encompassing any two of these values as endpoints.

In some embodiments, the size of the solid particles of the copolymer 106 may range from about 0.001 micrometers to about 1 micrometer (i.e., 1 nanometer to 1000 nanometers), or more specifically from about 1 nm to about 400 nm, or from about 10 nm to about 200 nm, or from about 10 nm to about 195 nm, or from about 10 nm to about 190 nm, or from about 10 nm to about 185 nm, or from about 10 nm to about 180 nm, or from about 10 nm to about 175 nm, or from about 10 nm to about 170 nm, or from about 10 nm to about 165 nm, or from about 20 nm to about 200 nm, or from about 20 nm to about 195 nm, or from about 20 nm to about 190 nm, or from about 20 nm to about 185 nm, or from about 20 nm to about 180 nm, or from about 20 nm to about 175 nm, or from about 20 nm to about 170 nm, or from about 20 nm to about 165 nm, or from about 25 nm to about 200 nm, or from about 25 nm to about 195 nm, or from about 25 nm to about 190 nm, or from about 25 nm to about 185 nm, or from about 25 nm to about 180 nm, or from about 25 nm to about 175 nm, or from about 25 nm to about 170 nm, or from about 25 nm to about 165 nm, or from about 30 nm to about 200 nm, or from about 30 nm to about 195 nm, or from about 30 nm to about 190 nm, or from about 30 nm to about 185 nm, or from about 30 nm to about 180 nm, or from about 30 nm to about 175 nm, or from about 30 nm to about 170 nm, or from about 30 nm to about 165 nm, or from about 33 nm to about 200 nm, or from about 33 nm to about 195 nm, or from about 33 nm to about 190 nm, or from about 33 nm to about 185 nm, or from about 33 nm to about 180 nm, or from about 33 nm to about 175 nm, or from about 33 nm to about 170 nm, or from about 33 nm to about 165 nm, or within any range encompassing any two of these values as endpoints.

In certain embodiments, within the composition 100, the ratio of the weight of the copolymer 106 to the volume of the liquid carrier (i.e., volume of the water miscible organic component 102 and the aqueous component 104 combined) may be from about 0.01 g/ml to about 0.1 g/ml, or from about 0.02 g/ml to about 0.1 g/ml, or from about 0.03 g/ml to about 0.1 g/ml, or from about 0.01 g/ml to about 0.09 g/ml, or from about 0.02 g/ml to about 0.09 g/ml, or from about 0.03 g/ml to about 0.09 g/ml, or from about 0.01 g/ml to about 0.08 g/ml, or from about 0.02 g/ml to about 0.08 g/ml, or from about 0.03 g/ml to about 0.08 g/ml, or from about 0.01 g/ml to about 0.07 g/ml, or from about 0.02 g/ml to about 0.07 g/ml, or from about 0.03 g/ml to about 0.07 g/ml, or from about 0.01 g/ml to about 0.06 g/ml, or from about 0.02 g/ml to about 0.06 g/ml, or from about 0.03 g/ml to about 0.06 g/ml, or within any range encompassing any two of these values as endpoints.

In some embodiments, the composition 100 may include up to 1%, up to 5%, up to 10%, or any other percentage by weight of said fluorinated copolymers that is suitable based on the particular copolymer selected, and can further include up to 0.1%, up to 0.5%, up to 1%, up to 5%, up to 10%, up to 15%, up to 25%, up to 35%, up to 45%, or any other percentage by weight of said fluorinated copolymers.

According to some embodiments, the composition 100 may optionally further include a radiopacity agent 108. The radiopacity agent 108 includes a material that does not allow radiation (e.g., X-ray, fluoroscopy) to pass through it, thus providing visibility, for example, during the introduction of the composition 100 into a patient's body under fluoroscopy of a certain wavelength. In certain embodiments, the composition 100 including the radiopacity agent 108 is visible within a patient's body under fluoroscopy with wavelength of from about 0.01 to about 10 nm.

In some embodiments, the radiopacity agent 108 includes tantalum, barium sulfate, iohexol, iodixanol, ioxaglic acid, lipiodol, and triiodobenzoic acid, iopamidol iopromide, and/or gold nanoparticles. In certain embodiments, the radiopacity agent 108 is selected from a group consisting of tantalum, barium sulfate, iohexol, iodixanol, ioxaglic acid, lipiodol, and triiodobenzoic acid.

In some embodiments, the composition 100 includes tantalum dispersed in the liquid carrier, forming a kinetically stable dispersion of both the copolymer 106 and the radiopacity agent 108 in the liquid carrier. In certain embodiments, the composition 100 includes iohexol suspended in the liquid carrier, forming a mixture of a kinetically stable dispersion of the copolymer 106 with a suspension of the radiopacity agent 108 in the liquid carrier.

The radiopacity agent 108 may include tantalum particles having a range of particle sizes, and the particular size of the particles is selected for the specific application it is being used for. In some embodiments, the radiopacity agent 108 may include tantalum particles having sizes ranging from about 0.5 micrometers to about 9 micrometers (i.e., 500 nanometers to 9000 nanometers), or more specifically from about 0.6 micrometers to about 9 micrometers, or from about 0.7 micrometers to about 9 micrometers, or from about 0.8 micrometers to about 9 micrometers, or from about 1 micrometer to about 9 micrometers, or from about 1.5 micrometers to about 9 micrometers, or from about 2 micrometers to about 9 micrometers, or from about 0.5 micrometers to about 8.5 micrometers, or from about 0.6 micrometers to about 8.5 micrometers, or from about 0.7 micrometers to about 8.5 micrometers, or from about 0.8 micrometers to about 8.5 micrometers, or from about 1 micrometer to about 8.5 micrometers, or from about 1.5 micrometers to about 8.5 micrometers, or from about 2 micrometers to about 8.5 micrometers, or from about 0.5 micrometers to about 8 micrometers, or from about 0.6 micrometers to about 8 micrometers, or from about 0.7 micrometers to about 8 micrometers, or from about 0.8 micrometers to about 8 micrometers, or from about 1 micrometer to about 8 micrometers, or from about 1.5 micrometers to about 8 micrometers, or from about 2 micrometers to about 8 micrometers, or from about 0.5 micrometers to about 7.5 micrometers, or from about 0.6 micrometers to about 7.5 micrometers, or from about 0.7 micrometers to about 7.5 micrometers, or from about 0.8 micrometers to about 7.5 micrometers, or from about 1 micrometer to about 7.5 micrometers, or from about 1.5 micrometers to about 7.5 micrometers, or from about 2 micrometers to about 7.5 micrometers, or from about 0.5 micrometers to about 7 micrometers, or from about 0.6 micrometers to about 7 micrometers, or from about 0.7 micrometers to about 7 micrometers, or from about 0.8 micrometers to about 7 micrometers, or from about 1 micrometer to about 7 micrometers, or from about 1.5 micrometers to about 7 micrometers, or from about 2 micrometers to about 7 micrometers, or from about 0.5 micrometers to about 6.5 micrometers, or from about 0.6 micrometers to about 6.5 micrometers, or from about 0.7 micrometers to about 6.5 micrometers, or from about 0.8 micrometers to about 6.5 micrometers, or from about 1 micrometer to about 6.5 micrometers, or from about 1.5 micrometers to about 6.5 micrometers, or from about 2 micrometers to about 6.5 micrometers, or from about 0.5 micrometers to about 6 micrometers, or from about 0.6 micrometers to about 6 micrometers, or from about 0.7 micrometers to about 6 micrometers, or from about 0.8 micrometers to about 6 micrometers, or from about 1 micrometer to about 6 micrometers, or from about 1.5 micrometers to about 6 micrometers, or from about 2 micrometers to about 6 micrometers, or from about 0.5 micrometers to about 5.5 micrometers, or from about 0.6 micrometers to about 5.5 micrometers, or from about 0.7 micrometers to about 5.5 micrometers, or from about 0.8 micrometers to about 5.5 micrometers, or from about 1 micrometer to about 5.5 micrometers, or from about 1.5 micrometers to about 5.5 micrometers, or from about 2 micrometers to about 5.5 micrometers, or from about 0.5 micrometers to about 5 micrometers, or from about 0.6 micrometers to about 5 micrometers, or from about 0.7 micrometers to about 5 micrometers, or from about 0.8 micrometers to about 5 micrometers, or from about 1 micrometer to about 5 micrometers, or from about 1.5 micrometers to about 5 micrometers, or from about 2 micrometers to about 5 micrometers, or within any range encompassing any two of these values as endpoints. In some embodiments, the radiopacity agent 108 may include tantalum particles having a spherical, elongated, or irregular shape.

In some embodiments, the radiopacity agent 108 may include iohexol particles having sizes ranging from about 0.001 micrometers to about 0.01 micrometers (i.e., 1 nanometer to 10 nanometers), or more specifically from about 1.5 nm to about 9 nm, or from about 2 nm to about 9 nm, or from about 2.5 nm to about 9 nm, or more specifically from about 1.5 nm to about 8 nm, or from about 2 nm to about 8 nm, or from about 2.5 nm to about 8 nm, or more specifically from about 1.5 nm to about 7 nm, or from about 2 nm to about 7 nm, or from about 2.5 nm to about 7 nm, or more specifically from about 1.5 nm to about 6 nm, or from about 2 nm to about 6 nm, or from about 2.5 nm to about 6 nm, or more specifically from about 1.5 nm to about 5 nm, or from about 2 nm to about 5 nm, or from about 2.5 nm to about 5 nm, or within any range encompassing any two of these values as endpoints.

In certain embodiments, within the composition 100, the ratio of the weight of the radiopacity agent 108 to the volume of the liquid carrier (i.e., volume of the water miscible organic component 102 and the aqueous component 104 combined) is from about 0.1 g/ml to about 0.75 g/ml, or from about 0.12 g/ml to about 0.75 g/ml, or from about 0.15 g/ml to about 0.75 g/ml, or from about 0.2 g/ml to about 0.75 g/ml, or from about 0.25 g/ml to about 0.75 g/ml, or from about 0.3 g/ml to about 0.75 g/ml, or from about 0.1 g/ml to about 0.5 g/ml, or from about 0.12 g/ml to about 0.5 g/ml, or from about 0.15 g/ml to about 0.5 g/ml, or from about 0.2 g/ml to about 0.5 g/ml, or from about 0.25 g/ml to about 0.5 g/ml, or from about 0.3 g/ml to about 0.5 g/ml, from about 0.1 g/ml to about 0.4 g/ml, or from about 0.12 g/ml to about 0.4 g/ml, or from about 0.15 g/ml to about 0.4 g/ml, or from about 0.2 g/ml to about 0.4 g/ml, or from about 0.25 g/ml to about 0.4 g/ml, or from about 0.3 g/ml to about 0.4 g/ml, or within any range encompassing any two of these values as endpoints.

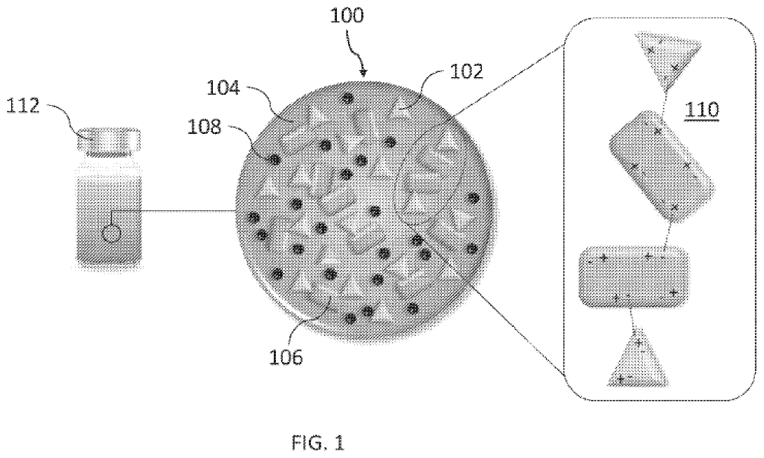

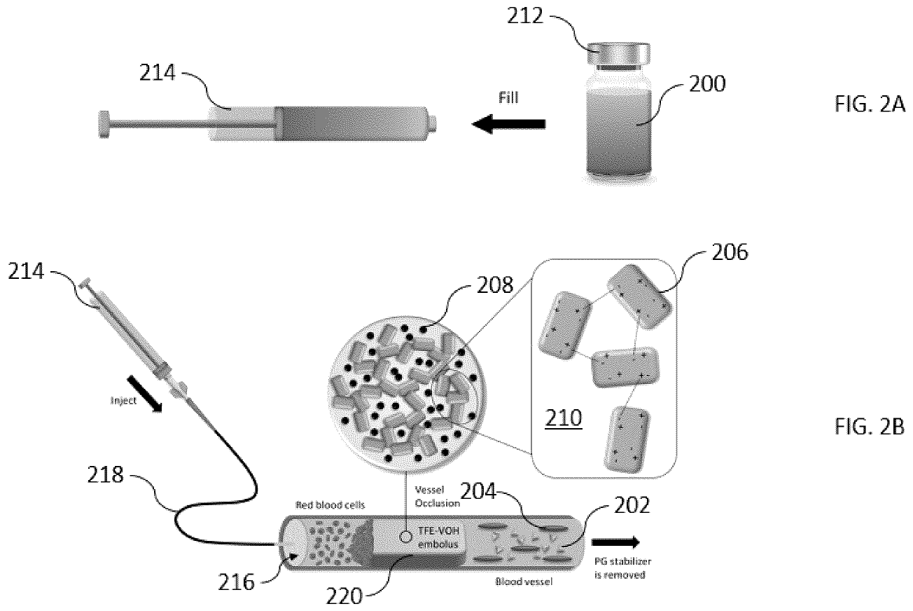

According to certain embodiments, and will be further discussed below with regard to FIG. 2, the composition 100 is configured to be injected into a target space to form a gel that occludes at least a portion of the space. The target space may be within a vessel, an organ, or a tissue of a patient. In some embodiments, the vessel is a vein or an artery having a diameter of less than 3 mm. In some embodiments, the vein or artery has a diameter of about 1 to about 10 mm. In some embodiments, the vein or artery has a diameter of about 3 to about 4 mm. In some embodiments, the vein or artery has a diameter of less than 10 mm. In certain embodiments, the target space may include vessels or pseudoaneurysm or aneurysms up to a diameter of 95 mm. During treatment, the composition 100 may be injected using a delivery device, including, but not limited to, a needle, a catheter, a microcatheter, or a syringe. In some embodiments, the composition 100 is an embolic agent used for an embolization procedure. In certain embodiments, for example when the composition 100 includes tantalum as the radiopacity agent 108, the composition 100 is a mixture of dispersion of the copolymer 106 and suspension of the radiopacity agent 108 within the liquid carrier, and is stirred or agitated before injection. After injection to a target space, the tetrafluoroethylene copolymer and the radiopacity agent are evenly distributed within the gel formed at the target space.

### IV. Applications

The compositions as disclosed herein may be used for occluding a lumen or a complex system of lumens in a living tissue/organism (e.g., liquid embolic therapy), filling an empty space or gap surrounded by living tissue (e.g., interstitial spaces in living tissue), and/or for use in tissue bulking applications.

IV-a. Embolic Therapy

Embolic therapy is a minimally invasive procedure performed to treat a variety of vascular pathologies, including but not limited to, preoperative management of hypervascularized tumors and arteriovenous malformations. Embolic therapy involves the intentional blockage or embolization of a cavity, blood vessel, or a system of blood vessels with an object to control or prevent blood flow to a vascular pathology (e.g., hypervascularized tumors and arteriovenous malformations). Hypervascularized tumors have abnormally large numbers of blood vessels providing circulation and are either malignant or benign. Arteriovenous malformations are abnormal connections between arteries and veins whose presence can lead to stroke and death. Hypervascularized tumors and arteriovenous malformations can occur in many areas of the body.

In addition, embolization can be used for treating fistulae, endoleaks, aneurysms (e.g., by filling or plugging the aneurismal sac), for embolizing a vessel to control bleeding due to lesions (e.g. organ bleeding, gastrointestinal bleeding, vascular bleeding, as well as bleeding associated with an aneurysm), for access closure, and for chronic total occlusion, and for the management of trauma, including embolization needed after traumatic injuries.

Embolic agents may be delivered to a designated area of the body through a syringe or a catheter device. The embolic agents can be permanent implants, biodegradable implants, or temporary implants removed by a second procedure. Embolic agents can be delivered in a solid form (such as a balloon on a catheter, or metal coils) or as a liquid form that hardens in vivo into a second form that can be a solid, a gel, or an intermediate state.

In the case of polymer based liquid embolic agents, after injection of the embolic agent to a treatment site and upon contact with blood or other bodily fluids, in a process referred to herein as hardening, the liquid carrier of the embolic agent diffuses away from the site, leaving behind a precipitated water-insoluble polymer that obstructs blood flow to the pathology. During the treatment process, the agent delivered undergoes a transition from a liquid state to a solid state or a gel state or an intermediate state, as a congealed mass or as multiple discrete particles, thereby embolizing the site.

Examples of polymer-based liquid embolic agents known in the art include poly(ethylene-vinyl alcohol) dissolved in dimethylsulfoxide (DMSO), poly(hydroxyethyl methacrylate) dissolved in ethanol, cyanoacrylate dissolved in lipiodol, and poly(lactide-glycolide) dissolved in N-methylpyrrolidone (NMP). DMSO, ethanol, lipiodol, and NMP have well known toxicities and side effects such as vasospasm, sclerosis, intoxication, and pain. Thus, their use must be carefully monitored when injected into a blood vessel, an organ, or other target site. In addition, these solvents release noisome vapors that can cause great discomfort for the patient and the surgical and medical staff. DMSO and NMP can also dissolve and damage common catheter and interventional materials, so these solvents require special and/or expensive catheters and other surgical equipment.

Embodiments of the present disclosure are directed to a class of fluorinated copolymers, such as TFE copolymers, that can be dispersed in liquid carriers that are of low toxicity to living tissue and that enable formation of compositions including kinetically stable dispersions of the polymeric particles within the liquid carrier, that may be used as an embolic agent. The liquid carrier as disclosed herein may include an aqueous component dissolved in a low toxicity water miscible organic component, and have reduced toxicity, side effects, and volatility compared to typical solvents used in the art. In addition, compositions disclosed herein have viscosity and low toxicity that allow for needle, catheter, and microcatheter delivery, and do not require specialized catheters because the described compositions do not or only inconsequentially damage commonly used catheters. As such, compositions according to embodiments disclosed herein can be delivered to anatomical target sites with fewer complications than embolic agents known in the art.

FIGS. 2A and 2B are schematic diagrams of an exemplary device configured to deliver a composition including a tetrafluoroethylene (TFE) copolymer dispersed therein, according to certain embodiments of the present disclosure.

According to some embodiments, a method includes providing a composition 200 in a vial 212, the composition 200 including a TFE copolymer uniformly distributed throughout a liquid carrier including a water miscible organic component 202 and an aqueous component 204. The composition 200 may optionally include a radiopacity agent dispersed or suspended in the liquid carrier. In some embodiments, the composition 200 is an embolic agent used for an embolization procedure.

As shown in FIG. 2B, the composition 200 may be transferred using a syringe 214 and introduced into a target space 216. Before the composition is introduced, an appropriately sized target space 216 is chosen for treatment, and a guidewire (not shown) may be tracked to the target space 216 for treatment.

In some embodiments, the composition 200 is introduced into a target space 216 using a microcatheter 218. The microcatheter 218 may have an outer diameter of from about 2.4 to about 2.8 French, an internal diameter of from about 0.5 mm to about 0.8 mm, and a usable length of from about 1500 to 1600 mm. In certain embodiments, the microcatheter 218 may have an outer diameter of from about 2.1 to about 2.8 French, an internal diameter of from about 0.4 mm to about 0.7 mm, and a usable length of from about 650 to 2200 mm. In other embodiments, the microcatheter 218 may have an outer diameter of from about 1.2 to about 4.6 French, an internal diameter of from about 0.2 mm to about 0.99 mm, and a usable length of from about 500 to 2200 mm. Alternatively, the composition 200 may be introduced into a target space 216 using a delivery device, including but not limited to, a needle, a catheter, or a syringe (not shown). After the guidewire is removed, the microcatheter 218 may be pre-filled with the liquid carrier including water miscible organic component 202 and the aqueous component 204 to aid with delivery of the composition 200.

In certain embodiments, the composition 200 includes a radiopacity agent to assist with visualization of the location of the composition 200 under fluoroscopic guidance during introduction. In some embodiments, the radiopacity agent includes tantalum suspended in the liquid carrier of the composition 200, and the composition 200 is stirred or agitated before the introducing step.

In some embodiments, the target space 216 may be an occlusion site of a patient. In certain embodiments, the occlusion site is within a vessel, an organ, or a tissue of the patient. In some embodiments, as will be discussed further in section IV-b below, the target space 216 may be a bulking site within a vessel, an organ, or a tissue of a patient. In some embodiments, the vessel is a vein or an artery having a diameter of less than 3 mm. In some embodiments, the vessel is a vein or an artery having a diameter of about 3 mm to about 10 mm. In certain embodiments, the vessel is a vein or an artery having a diameter of about 3 mm to about 4 mm. In certain embodiments, the target space 216 may be a vessel, pseudoaneurysm, or aneurysm having a diameter of larger than 3 mm, or 5 mm, or 10 mm, or 20 mm, or 40 mm, or 60 mm, or 80 mm, or 90 mm, or 95 mm, or within any range encompassing any two of these values as endpoints.

After the composition 200 is introduced to the target space 216 and upon contact of the composition 100 with blood or other bodily fluids, the water miscible organic component 202 diffuses into the blood stream or bodily fluid, thus forming a gel 220 including the copolymer 206 at the target space 216. Diffusion of the water miscible organic component 202 from the composition 200 forms a solid or gel-like embolic mass as a congealed mass or as multiple, discrete particles. In certain embodiments, the TFE copolymer 206 and/or the radiopacity agent 208 from the composition 200 are evenly distributed within the gel 220 formed at the target space 216. As shown, more physical crosslinks 210 (e.g., hydrogen bonds) are formed in between the copolymer particles 206 upon diffusion of the organic component 202 from the composition 200, thus hardening the composition 200 to form a gel 220 that occludes the target space 216.

After the introduction of the composition 200 to the target space 216, serial angiograms may be used from the guide catheter (not shown) to inject contrast to assess successful occlusion of the target space 216. Additional embolic materials may be injected as needed to achieve complete occlusion of the target space 216. After successful occlusion, the guide catheter (not shown) may be removed, and a final angiogram may be performed to assess the completion of treatment.

IV-b. Filler

Filling gaps or void spaces can have many applications. Filling gaps or void spaces can occur external to the body or in vivo. In an embodiment, the compositions described herein can facilitate treatment of an endoleak or to facilitate sealing or adhering at least a portion of the outer surface of a graft, stent, or stent graft to the surrounding vessel or a neighboring graft, stent, or stent graft. In some embodiments, the compositions described herein can be used as a filler at a bulking site of a patient. In certain embodiments, the bulking site is within a vessel, an organ, or a tissue of a patient.

Described compositions can be combined with other materials for alternate properties or morphologies and be used for coating or filling an interstitial space. For example, described compositions can be mixed with a bioabsorbable material. As bioabsorption occurs, voids can form in the hardened compositions creating a porous or sponge-like polymeric material. In various embodiments, the void spaces can be sites where tissue ingrowth can occur. Examples of bioabsorbable material that may be mixed with the compositions described herein includes poly(lactic acid-co-glycolic acid) (PLA-PGA) adjusted in the desired ratio to achieve the desired rate of biological absorption. Other potentially useful bioabsorbable materials include polyglycolic acid (PGA), poly-L-lactic acid (PLA), polydiaoxanone (PDS), polyhydroxybutyrate, copolymers of hydroxybutyrate and hydroxyvalerate, copolymers of lactic acid and E-caprolactone, oxidized regenerated cellulose and various forms of collagen. Another embodiment includes mixing the compositions described herein with a poly(glycolide-co-trimethylene carbonate) tri-block copolymer (PGA:TMC), e.g., a non-woven, bioabsorbable web material. The proportions of this or any other selected copolymer or blends of polymers can be adjusted to achieve the desired absorption rate.

In another embodiment, the described compositions can be mixed with a leachable pore-forming material that produces pores during the drying or hardening of the TFE copolymer compositions to form a sponge-like material. In an embodiment, said leachable pore-forming agent can be combined before, during, or after delivery of the compositions in vivo. A leachable pore-forming agent can include but is not limited to particles of sodium chloride, glucose, sucrose, bioabsorbable material, collagen, albumin, hydroxyapatite, lipids, polyethylene glycol, polyvinyl alcohol, and the like. Further embodiments described herein include systems or kits including compositions as described herein and a delivery device. In some embodiments, delivery devices include an implantation guide, which facilitates delivery of said compositions to an implantation site by providing a delivery path. In other embodiments, delivery devices include an implantation guide and a translating and/or rotating member, wherein the translating and/or rotating member facilitates translation of said composition along the delivery path defined by the implantation guide. Translating member embodiments may include a syringe, an implantation piston member, or any other device that facilitates translation of said composition along the delivery path.

IV-b. Methods of Treatment

According to some embodiments, the compositions described herein may be used in medical procedures for distal embolization in peripheral arterial vasculature, including treatment of hemorrhage and hypervascular tumors. The operational sequence for using such compositions may include device preparation, microcatheter preparation, and device deployment through a microcatheter.

In some embodiments, the method may include obtaining the composition from a storage location and delivering it to a treatment area. This may include selecting the appropriate composition, opening the package, and inspecting for damage. In certain embodiments, the composition vial may be attached to a shaker for a predetermined duration to ensure homogeneous mixing of the contents in the vial, particularly when the composition includes suspended particles such as radiopacity agents.

In some aspects, the method may include transferring provided syringes to a sterile field, with the composition vials remaining external to the sterile field. During microcatheter preparation, a primer solution may be obtained and a tamper evident lid may be removed. The primer solution may be aspirated from the vial using a supplied syringe with an attached needle. The needle may then be removed from the syringe, the syringe may be attached to the microcatheter, and the microcatheter may be primed.

During composition deployment, the composition vial may be obtained and the tamper evident lid may be removed. The composition material may be aspirated from the vial using a supplied syringe with an attached needle. In certain embodiments, the needle may be removed from the syringe, the syringe may be attached to the microcatheter, and the volume of the microcatheter may be filled with the composition. The composition may then be deployed to the target site until occlusion of the target region is achieved.

In some embodiments, following the procedure, the packaging and any composition remnants may be discarded using standard practices for contaminated waste. In certain aspects, following the procedure, the patient may be provided with documentation for future reference, which may contain information regarding the implanted material for use in subsequent medical consultations or procedures.

In accordance with embodiments, the target site for the deposition of the composition is adjacent to a distal tip or dispensing port of a delivery device that is operable to dispense the composition. Examples of such delivery devices include, but are not limited to a needle, a catheter, a microcatheter, and a syringe. In other embodiments, the target site for the deposition of the composition is distal or downstream a distance away from the delivery device distal tip or dispensing port. In these embodiments, the composition is conveyed from the delivery device to a downstream target site by fluid flow, such as blood flow by way of example, otherwise referred to as distal embolization. Distal embolization, or the distal embolization method of treatment, allows for, by way of example, the deposition of the composition in vascular wherein the target site is not accessible by the delivery device but is located downstream of the delivery device. In accordance with an embodiment, deploying the composition includes deploying the composition from the delivery device operable to deliver the composition to a distal target site a distance from the delivery device so as to achieve occlusion of the target site. In accordance with another embodiment, deploying the composition includes deploying the composition from the delivery device operable to deliver the composition to a distal target site a distance from the delivery device so as to achieve occlusion of the target site in peripheral arterial vasculature. In accordance with another embodiment, the distal embolization as described above is for treatment of hemorrhage. In accordance with another embodiment, the distal embolization as described above is for treatment of hypervascular tumors.

### V. Methods of Making

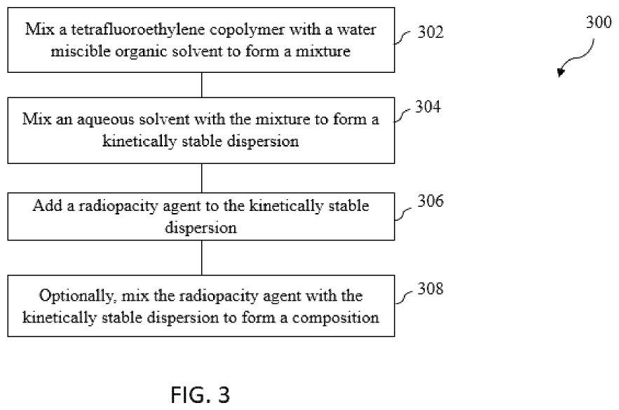

FIG. 3 is a flow diagram illustrating a process 300 of making a composition including a tetrafluoroethylene (TFE) copolymer dispersed therein, according to certain embodiments of the present disclosure. One or more steps of the process 300 are optional and/or can be modified by one or more steps of other embodiments described herein. Additionally, one or more steps of other embodiments described herein may be added to the process 300.

At step 302, in some embodiments, the process 300 includes mixing a TFE copolymer with a water miscible organic component to form a mixture. At step 304, in some embodiments, the process 300 includes mixing an aqueous component with the mixture to form a kinetically stable dispersion, the dispersion including the TFE copolymers uniformly distributed throughout a liquid carrier including the water miscible organic component and the aqueous component. In some embodiments, step 302 and step 304 may be combined into one step, such that the TFE copolymer, the water miscible organic component, and the aqueous component are mixed simultaneously to form a kinetically stable dispersion.

In some embodiments, the mixing steps 302 and/or 304 are conducted under mechanical agitation at a temperature of from about 50° C. to about 120° C., or from about 55° C. to about 120° C., or from about 60° C. to about 120° C., or from about 65° C. to about 120° C., or from about 50° C. to about 115° C., or from about 55° C. to about 115° C., or from about 60° C. to about 115° C., or from about 65° C. to about 115° C., or from about 50° C. to about 110° C., or from about 55° C. to about 110° C., or from about 60° C. to about 110° C., or from about 65° C. to about 110° C., or from about 50° C. to about 105° C., or from about 55° C. to about 105° C., or from about 60° C. to about 105° C., or from about 65° C. to about 105° C., or from about 50° C. to about 100° C., or from about 55° C. to about 100° C., or from about 60° C. to about 100° C., or from about 65° C. to about 100° C., or within any range encompassing any two of these values as endpoints.

In certain embodiments, the mixing steps 302 and/or 304 are conducted under mechanical agitation for a duration of from about 0.5 hour to about 24 hours, or from about 0.5 hour to about 16 hours, or from about 0.5 hour to about 12 hours, or from about 1 hour to about 24 hours, or from about 1 hour to about 16 hours, or from about 1 hour to about 12 hours, or from about 2 hours to about 24 hours, or from about 2 hours to about 16 hours, or from about 2 hours to about 12 hours, or within any range encompassing any two of these values as endpoints. In certain embodiments, the mechanical agitation used during the mixing steps 302 and/or 304 may include use of a tumbling fixture or shaker plate, via stirring, or a combination thereof.

At step 306, in some embodiments, the process 300 includes adding a radiopacity agent to the kinetically stable dispersion to form a composition. In some embodiments, the formed composition includes the TFE copolymers dispersed (e.g., uniformly distributed) in the liquid carrier including the water miscible organic component and the aqueous component, and the radiopacity agent particles (e.g., tantalum) suspended in the liquid carrier.

At step 308, in some embodiments, the process 300 may optionally include mixing the radiopacity agent with the kinetically stable dispersion to form the composition. In some embodiments, the mixing step 308 may be conducted under mechanical agitation at a temperature of from about 10° C. to about 100° C., or from about 15° C. to about 100° C., or from about 20° C. to about 100° C., or from about 25° C. to about 100° C., or from about 30° C. to about 100° C., or from about 10° C. to about 85° C., or from about 15° C. to about 85° C., or from about 20° C. to about 85° C., or from about 25° C. to about 85° C., or from about 30° C. to about 85° C., or from about 10° C. to about 70° C., or from about 15° C. to about 70° C., or from about 20° C. to about 70° C., or from about 25° C. to about 70° C., or from about 30° C. to about 70° C., or within any range encompassing any two of these values as endpoints.

In certain embodiments, the mixing step 308 is conducted under mechanical agitation for a duration of from about 0.5 hour to about 24 hours, or from about 0.5 hour to about 16 hours, or from about 0.5 hour to about 12 hours, or from about 1 hour to about 24 hours, or from about 1 hour to about 16 hours, or from about 1 hour to about 12 hours, or from about 2 hours to about 24 hours, or from about 2 hours to about 16 hours, or from about 2 hours to about 12 hours, or within any range encompassing any two of these values as endpoints. In certain embodiments, the mechanical agitation used during the mixing step 308 may include use of a tumbling fixture or shaker plate, via stirring, or a combination thereof. In some embodiments, the formed composition includes both the TFE copolymers and the radiopacity agent particles (e.g., iohexol) dispersed (e.g., uniformly distributed) in the liquid carrier including the water miscible organic component and the aqueous component.

### VI. Test Methods

Various techniques as discussed in this section were used to characterize embodiments of the compositions as described herein.

VI-a. Dynamic Light Scattering (DLS)

DLS is a technique that measures the size of particles in a liquid by analyzing how light scatters from them. Samples need to be translucent liquids. Thus, compositions that are not translucent (e.g., having particles suspended therein) cannot be measured using DLS.

Samples of the compositions were pipetted into a disposable, plastic cuvette, and analyzed using a Malvern Zetasizer Ultra instrument (i.e., a Zeta Potential Analyzer) Model ZSU5700. After the sample was loaded into the instrument, a light shield was placed over the sample to block out any additional light. To analyze an exemplary sample composition synthesized and described herein at room temperature, the refractive index for the material was set to 1.46, the refractive index for the dispersant was set to 1.33, and the viscosity was set to 12 mPa*s. The settings for the refractive indexes and viscosity may be different depending on the concentration of the copolymer in the sample as well as the ratio of different components within the liquid carrier. The temperature of the instrument was set to be 25° C. for the analysis, and the method builder was multi-angle dynamic light scattering (MADLS). ZS Explorer software was used to collect the data, and results were exported to an excel spreadsheet and further analyzed.

VI-b. Transmission Electron Microscopy (TEM) Imaging

TEM is an electron microscopy technique that uses a beam of high energy electrons to obtain a high resolution image through the detection of transmitted and scattered electrons of a thin specimen, about 100 nm thick. The transmitted electrons carry information about the density and crystalline state of the sample, while the scattered electrons provide information about the sample composition.

One process to prepare samples for TEM, especially helpful for samples in a fluid, is vitrification. The vitrification process quickly freezes a liquid sample thus preserving the sample close to its natural state. The goal is to freeze the sample without forming ice crystals which might damage the sample. The vitrification process includes placing a droplet of a sample suspension on to a carbon coated TEM grid, blotting the grid with filter paper to remove the excess fluid until a thin film remains, then quickly plunge freezing the grid with the thin film into a cryogen to create a thin layer of amorphous ice. The sample is then kept frozen and transferred in the TEM for imaging and analysis. This complex sample preparation process may be aided using automated systems (e.g., Vitrobot™).

While particular embodiments of the present disclosure have been illustrated and described herein, the present disclosure should not be limited to such illustrations and descriptions. It should be apparent that changes and modifications could be incorporated and embodied as part of the present disclosure within the scope of the following claims. The following examples are further offered to illustrate the present disclosure.

### EXAMPLES

### Example 1: Synthesis of TFE-VOH Copolymer Dispersed in PG/PBS Solution

A dispersion of TFE-VOH copolymer particles uniformly distributed throughout a PG/PBS liquid carrier was prepared.

3 grams of TFE-VOH (VOH:TFE (mol %)=80:20, size=135 nm, MW=230,000) was weighed and added to 60 grams (or mL) of PG and 40 grams (or mL) of PBS. The density of PG and PBS are both roughly 1 g/mL, and therefore gram and mL may be used interchangeably to describe the PG/PBS solution. The resulting mixture was heated in an oven at 80° C. for 24 hours to make a composition having TFE-VOH copolymer particles dispersed in a PG/PBS solution.

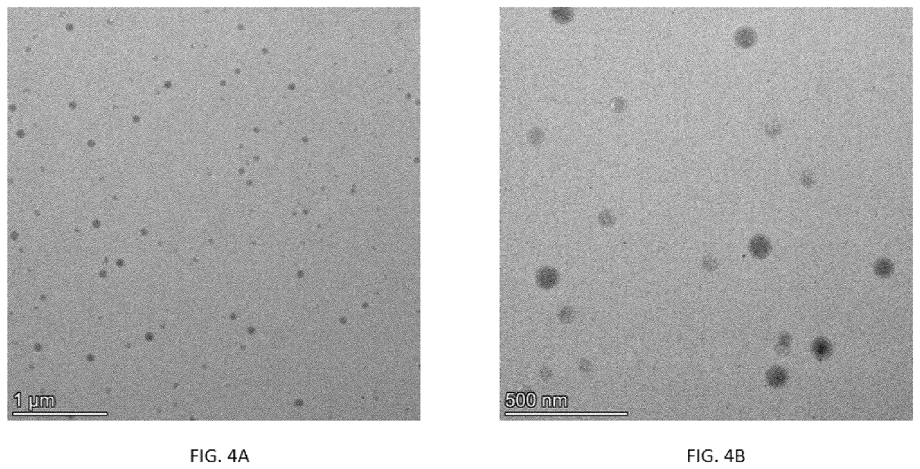

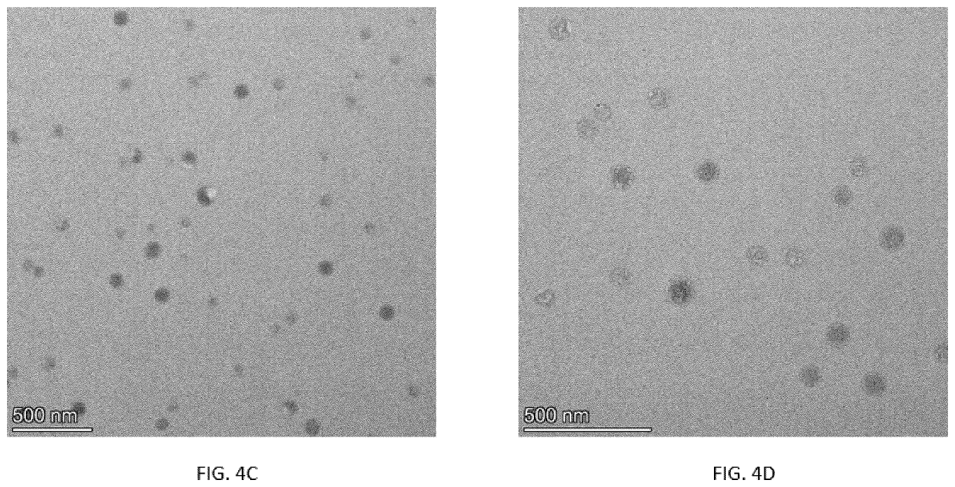

TEM images were taken of the resulting composition using imaging methods described above in the Test Method section. FIGS. 4A-4C include TEM images showing various locations of the sample at different scales. As shown in FIGS. 4A-4C, the resulting composition includes TFE-VOH copolymer particles uniformly distributed throughout the PG/PBS solution, thus forming a dispersion.

### Example 2: Kinetic Stability of TFE-VOH Copolymer Dispersed in PG/PBS Solution

Two compositions of TFE-VOH copolymer particles uniformly distributed in PG/PBS solutions (liquid carrier) were prepared.

“Aged Sample” with Iohexol Preparation: 0.6 grams of TFE-VOH same as the copolymer used in Example 1 was weighed and transferred to a 40 mL glass vial. 12 mL of PG was pipetted and added into the vial. 8 mL of PBS was pipetted and added into the vial. The vial was sealed and cooked overnight at 85° C. in a tumbling fixture to form a first dispersion having the copolymers dispersed within the PG/PBS solution. 6.0 grams of iohexol was weighed and transferred to the vial containing the first dispersion. The vial was sealed and cooked overnight at 50° C. in a tumbling fixture at 100 rpm, forming a second dispersion including both the copolymer particles and iohexol particles dispersed in the PG/PBS solution. The vial was then removed from the oven and placed in a dry location to age for about 2 years without stirring or agitation to form an “Aged Sample”.

“Fresh Sample” with Iohexol Preparation: 0.6 grams of TFE-VOH same as the copolymer used in Example 1 was weighed and transferred to a 40 mL glass vial. 12 mL of PG was pipetted and added into the vial. The vial was sealed and cooked overnight at 85° C. in a tumbling fixture. The vial was then removed from the oven, and 8 mL of PBS was pipetted and added into the vial. The vial was sealed again and cooked overnight at 85° C. in a tumbling fixture to form a dispersion including the copolymer particles dispersed in the PG/PBS solution. The vial was then removed from the oven and placed in a dry location until testing.

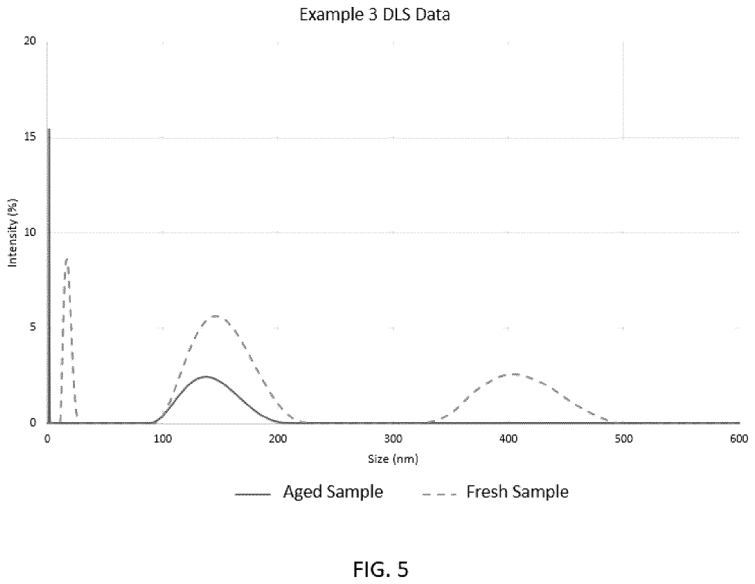

DLS analyses were conducted on the “Aged Sample” about 2 years after it was made, and on the “Fresh Sample” about 3 weeks after it was made. The plotted data from the DLS analyses is shown in FIG. 5, with the solid line showing DLS data of the Aged Sample, and the dashed line showing DLS data of the Fresh Sample.

As shown, the Aged Sample data showed no aggregation of the TFE-VOH nanoparticles forming larger microparticles. Neither sample showed a peak past 500 nm meaning that each sample was a stable colloid. Even though the Fresh Sample has a second peak at 400 nm, the data from each sample supports a conclusion that both samples were stable colloids that were not aggregating to form larger particles in the micrometer range. Therefore, this data shows that the Aged Sample did not form larger particles after a shelf life of over 2 years, the addition of the radiopacity agent (iohexol) did not affect particle formation, the samples did not contain particles larger than 500 nm before or after aging, and the samples were stable dispersions both before and after aging.

### Examples 3-7: Synthesis of TFE-VOH Copolymer Dispersed in PG/PBS Solution and Tantalum Suspended in PG/PBS Solution

A dispersion of TFE-VOH copolymer particles uniformly distributed throughout a PG/PBS solution (liquid carrier) was prepared using the method described in Example 17.

0.6 grams of TFE-VOH copolymer same as the copolymer used in Example 1 was weighed and added to 12 grams of PG and 8 grams of PBS. The resulting mixture was heated in an oven at 80° C. for 24 hours to make a dispersion of the copolymers within the PG/PBS solution to form a dispersion including the polymer particles dispersed in the PG/PBS solution. Different amounts of tantalum particles were then added to the dispersion as listed in Table 3 to achieve different loading of tantalum in each sample for Examples 3-7.

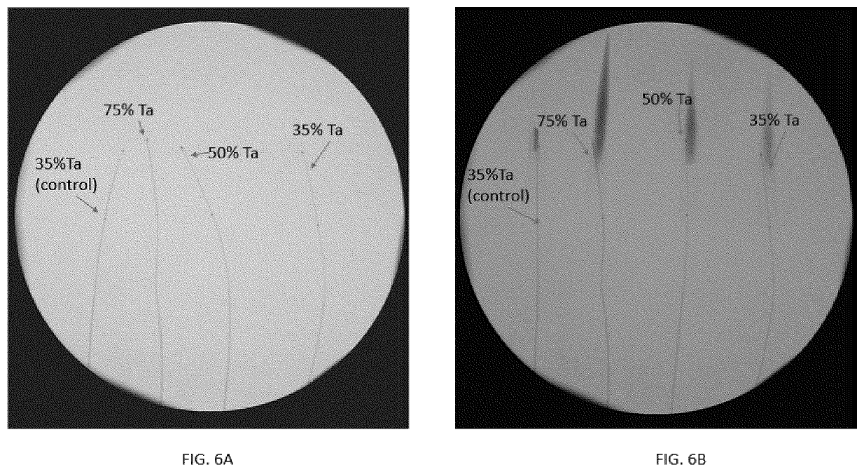

Fluoroscopy images were taken of samples made in Example 5, Example 6, and Example 7, and results are shown in FIGS. 6A and 6B. The C-Arm model used to take the fluoroscopy images was the Philips Veradius 1.2 Neo (Asset 20015710) with attenuation plate (Asset 20037675-84220) kV set to Auto, continuous X mode, speed 12, II size 12, object to detector distance ˜350 mm, and source to detector distance 30.125. As shown, compositions having a higher Tantalum loading are more visible under fluoroscopy.

TABLE 2
Tantalum (Ta) Loading for Examples 3-7

| | TFE-VOH | PG | PBS | Tantalum | Ta Loading |
|---|---|---|---|---|---|
| | (g) | (mL) | (mL) | (g) | (w/v) |
| Example 3 | 0.6 | 12 | 8 | 2.32 | 11.6% |
| Example 4 | 0.6 | 12 | 8 | 4.64 | 23.2% |
| Example 5 | 0.6 | 12 | 8 | 7 | 35% |
| Example 6 | 0.6 | 12 | 8 | 10 | 50% |
| Example 7 | 0.6 | 12 | 8 | 15 | 75% |

### Example 8: Stepwise Synthesis of TFE-VOH Copolymer and Iohexol Particles Dispersed in PG/PBS Solution

A dispersion of TFE-VOH copolymer and Iohexol particles uniformly distributed throughout a PG/PBS solution (liquid carrier) was prepared.

0.6 grams of TFE-VOH same as the copolymer used in Example 1 was weighed and transferred to a 40 mL glass vial. 12 mL of PG was pipetted and added into the vial. The vial was sealed and cooked overnight at 80° C. on a shaker plate. The vial was then removed from the shaker plate, and 8 mL of PBS was pipetted and added into the vial. The vial was sealed again and cooked overnight at 80° C. on a shaker plate to form a dispersion including the polymer particles dispersed in the PG/PBS solution.

6.0 grams of Iohexol was weighed and added to the dispersion in the vial. The vial was shaken at about 100 rpm at 50° C. overnight to form a composition having both the TFE-VOH copolymer and the iohexol particles dispersed in the PG/PBS solution. 1.5 mL of the composition was then pipetted into a smaller vial for storage and transferring.

### Example 9: Stepwise Synthesis of TFE-VOH Copolymer Dispersed in PG/PBS Solution And Tantalum Particles Suspended in PG/PBS Solution

A dispersion of TFE-VOH copolymer uniformly distributed throughout a PG/PBS solution (liquid carrier) and tantalum particles suspended in the liquid carrier was prepared.

0.6 grams of TFE-VOH same as the copolymer used in Example 1 was weighed and transferred to a 40 mL glass vial. 12 mL of PG was pipetted and added into the vial. The vial was sealed and cooked overnight at 80° C. on a shaker plate. The vial was then removed from the shaker plate, and 8 mL of PBS was pipetted and added into the vial. The vial was sealed again and cooked overnight at 80° C. on a shaker plate to form a dispersion including the polymer particles dispersed in the PG/PBS solution.

0.525 grams of Tantalum was weighed and added to the dispersion in the vial to form a composition having the TFE-VOH copolymer dispersed in the PG/PBS solution and tantalum particles suspended in solution. 1.5 mL of the composition was then pipetted into a smaller vial for storage and transferring.

### Example 10: Melting-Pot Synthesis of TFE-VOH Copolymer and Iohexol Particles Dispersed in PG/PBS Solution

A dispersion of TFE-VOH copolymer and Iohexol particles uniformly distributed throughout a PG/PBS solution (liquid carrier) was prepared.

0.6 grams of TFE-VOH same as the copolymer used in Example 1 was weighed and transferred to a 40 mL glass vial. 12 mL of PG was pipetted and added into the vial. 8 mL of PBS was pipetted and added into the vial. The vial was sealed and cooked overnight at 80° C. on a shaker plate to form a dispersion including the polymer particles dispersed in the PG/PBS solution.

6.0 grams of Iohexol was weighed and added to the dispersion in the vial. The vial was shaken at about 100 rpm at 50° C. overnight to form a composition having both the TFE-VOH copolymer and the iohexol particles dispersed in the PG/PBS solution. 1.5 mL of the composition was then pipetted into a smaller vial for storage and transferring.

### Example 11: Melting-Pot Synthesis of TFE-VOH Copolymer Dispersed in PG/PBS Solution and Tantalum Particles Suspended in PG/PBS Solution

A dispersion of TFE-VOH copolymer uniformly distributed throughout a PG/PBS solution (liquid carrier) and tantalum particles suspended in the liquid carrier was prepared.

0.6 grams of TFE-VOH same as the copolymer used in Example 1 was weighed and transferred to a 40 mL glass vial. 12 mL of PG was pipetted and added into the vial. 8 mL of PBS was pipetted and added into the vial. The vial was sealed and cooked overnight at 80° C. on a shaker plate to form a dispersion including the polymer particles dispersed in the PG/PBS solution.

0.525 grams of Tantalum was weighed and added to the dispersion in the vial to form a composition having the TFE-VOH copolymer dispersed in the PG/PBS solution and tantalum particles suspended in solution. 1.5 mL of the composition was then pipetted into a smaller vial for storage and transferring.

### Example 12: Stepwise Synthesis of TFE-VOH Copolymer and Iohexol Particles Dispersed in PG/PBS Solution

A dispersion of TFE-VOH copolymer and Iohexol particles uniformly distributed throughout a PG/PBS solution (liquid carrier) was prepared.

0.6 grams of TFE-VOH same as the copolymer used in Example 1 was weighed and transferred to a 40 mL glass vial. 12 mL of PG was pipetted and added into the vial. A stir bar was added to the vial. The vial was sealed and cooked overnight at 80° C. on a stir plate. The vial was then removed from the stir plate, and 8 mL of PBS was pipetted and added into the vial. The vial was sealed again and cooked overnight at 80° C. on a stir plate to form a dispersion including the polymer particles dispersed in the PG/PBS solution.

6.0 grams of Iohexol was weighed and added to the dispersion in the vial. The vial was stirred at 50° C. overnight to form a composition having both the TFE-VOH copolymer and the iohexol particles dispersed in the PG/PBS solution. 1.5 mL of the composition was then pipetted into a smaller vial for storage and transferring.

### Example 13: Stepwise Synthesis of TFE-VOH Copolymer Dispersed in PG/PBS Solution and Tantalum Particles Suspended in PG/PBS Solution

A dispersion of TFE-VOH copolymer uniformly distributed throughout a PG/PBS solution (liquid carrier) and tantalum particles suspended in the liquid carrier was prepared.

0.6 grams of TFE-VOH same as the copolymer used in Example 1 was weighed and transferred to a 40 mL glass vial. 12 mL of PG was pipetted and added into the vial. A stir bar was added to the vial. The vial was sealed and cooked overnight at 80° C. on a stir plate. The vial was then removed from the shaker plate, and 8 mL of PBS was pipetted and added into the vial. The vial was sealed again and cooked overnight at 80° C. on a stir plate to form a dispersion including the polymer particles dispersed in the PG/PBS solution.

0.525 grams of Tantalum was weighed and added to the dispersion in the vial to form a composition having the TFE-VOH copolymer dispersed in the PG/PBS solution and tantalum particles suspended in solution. 1.5 mL of the composition was then pipetted into a smaller vial for storage and transferring.

### Example 14: Melting-Pot Synthesis of TFE-VOH Copolymer and Iohexol Particles Dispersed in PG/PBS Solution

A dispersion of TFE-VOH copolymer and Iohexol particles uniformly distributed throughout a PG/PBS solution (liquid carrier) was prepared.

0.6 grams of TFE-VOH same as the copolymer used in Example 1 was weighed and transferred to a 40 mL glass vial. A stir bar was added to the vial. 12 mL of PG was pipetted and added into the vial. 8 mL of PBS was pipetted and added into the vial. The vial was sealed and cooked overnight at 80° C. on a stir plate to form a dispersion including the polymer particles dispersed in the PG/PBS solution.

6.0 grams of Iohexol was weighed and added to the dispersion in the vial. The vial was stirred at 50° C. overnight to form a composition having both the TFE-VOH copolymer and the iohexol particles dispersed in the PG/PBS solution. 1.5 mL of the composition was then pipetted into a smaller vial for storage and transferring.

### Example 15: Melting-Pot Synthesis of TFE-VOH Copolymer Dispersed in PG/PBS Solution and Tantalum Particles Suspended in PG/PBS Solution

A dispersion of TFE-VOH copolymer uniformly distributed throughout a PG/PBS solution (liquid carrier) and tantalum particles suspended in the liquid carrier was prepared.

0.6 grams of TFE-VOH same as the copolymer used in Example 1 was weighed and transferred to a 40 mL glass vial. A stir bar was added to the vial. 12 mL of PG was pipetted and added into the vial. 8 mL of PBS was pipetted and added into the vial. The vial was sealed and cooked overnight at 80° C. on a stir plate to form a dispersion including the polymer particles dispersed in the PG/PBS solution.

0.525 grams of Tantalum was weighed and added to the dispersion in the vial to form a composition having the TFE-VOH copolymer dispersed in the PG/PBS solution and tantalum particles suspended in solution. 1.5 mL of the composition was then pipetted into a smaller vial for storage and transferring.

### Example 16: Stepwise Synthesis of TFE-VOH Copolymer and Iohexol Particles Dispersed in PG/PBS Solution

A dispersion of TFE-VOH copolymer and Iohexol particles uniformly distributed throughout a PG/PBS solution (liquid carrier) was prepared.

0.6 grams of TFE-VOH same as the copolymer used in Example 1 was weighed and transferred to a 40 mL glass vial. 12 mL of PG was pipetted and added into the vial. The vial was sealed and cooked overnight at 85° C. on a tumbling fixture. The vial was then removed from the oven and tumbling fixture, and 8 mL of PBS was pipetted and added into the vial. The vial was sealed again and cooked overnight at 85° C. on a tumbling fixture to form a dispersion including the polymer particles dispersed in the PG/PBS solution.

6.0 grams of Iohexol was weighed and added to the dispersion in the vial. The vial was on the tumbling fixture at 50° C. at about 100 rpm overnight to form a composition having both the TFE-VOH copolymer and the iohexol particles dispersed in the PG/PBS solution. 1.5 mL of the composition was then pipetted into a smaller vial for storage and transferring.

### Example 17: Stepwise Synthesis of TFE-VOH Copolymer Dispersed in PG/PBS Solution and Tantalum Particles Suspended in PG/PBS Solution

A dispersion of TFE-VOH copolymer uniformly distributed throughout a PG/PBS solution (liquid carrier) and tantalum particles suspended in the liquid carrier was prepared.

0.6 grams of TFE-VOH same as the copolymer used in Example 1 was weighed and transferred to a 40 mL glass vial. 12 mL of PG was pipetted and added into the vial. The vial was sealed and cooked overnight at 85° C. on a tumbling fixture. The vial was then removed from the oven and tumbling fixture, and 8 mL of PBS was pipetted and added into the vial. The vial was sealed again and cooked overnight at 85° C. on a tumbling fixture to form a dispersion including the polymer particles dispersed in the PG/PBS solution.

0.525 grams of Tantalum was weighed and added to the dispersion in the vial to form a composition having the TFE-VOH copolymer dispersed in the PG/PBS solution and tantalum particles suspended in solution. 1.5 mL of the composition was then pipetted into a smaller vial for storage and transferring.

### Example 18: Melting-Pot Synthesis of TFE-VOH Copolymer and Iohexol Particles Dispersed in PG/PBS Solution

A dispersion of TFE-VOH copolymer and Iohexol particles uniformly distributed throughout a PG/PBS solution (liquid carrier) was prepared.

0.6 grams of TFE-VOH same as the copolymer used in Example 1 was weighed and transferred to a 40 mL glass vial. 12 mL of PG was pipetted and added into the vial. 8 mL of PBS was pipetted and added into the vial. The vial was sealed and cooked overnight at 85° C. on a tumbling fixture to form a dispersion including the polymer particles dispersed in the PG/PBS solution.

6.0 grams of Iohexol was weighed and added to the dispersion in the vial. The vial was placed on a tumbling fixture and tumbled at about 100 rpm at 50° C. overnight to form a composition having both the TFE-VOH copolymer and the iohexol particles dispersed in the PG/PBS solution. 1.5 mL of the composition was then pipetted into a smaller vial for storage and transferring.

### Example 19: Melting-Pot Synthesis of TFE-VOH Copolymer Dispersed in PG/PBS Solution and Tantalum Particles Suspended in PG/PBS Solution

A dispersion of TFE-VOH copolymer uniformly distributed throughout a PG/PBS solution (liquid carrier) and tantalum particles suspended in the liquid carrier was prepared.

0.6 grams of TFE-VOH same as the copolymer used in Example 1 was weighed and transferred to a 40 mL glass vial. 12 mL of PG was pipetted and added into the vial. 8 mL of PBS was pipetted and added into the vial. The vial was sealed and cooked overnight at 85° C. on a tumbling fixture to form a dispersion including the polymer particles dispersed in the PG/PBS solution.

0.525 grams of Tantalum was weighed and added to the dispersion in the vial to form a composition having the TFE-VOH copolymer dispersed in the PG/PBS solution and tantalum particles suspended in solution. 1.5 mL of the composition was then pipetted into a smaller vial for storage and transferring.

### Example 20: Embolization Treatment Procedure

20 mL of the dispersion of TFE-VOH copolymer uniformly distributed throughout a PG/PBS solution (liquid carrier) made according to Example 1 was pipetted and added into the vial. 6.0 grams of Iohexol was weighed and added to the dispersion in the vial. The vial was stirred at 50° C. overnight to form a composition including both the TFE-VOH copolymer and iohexol particles dispersed in the PG/PBS solution (liquid carrier).

The composition was delivered into a porcine kidney under fluoroscopy guidance according to the below procedure.

1. A guidewire was tracked to the treatment site.

2. An appropriately sized target was chosen for treatment.

3. A microcatheter was advanced over a marker wire (or similar as needed) to the treatment site.

4. The marker wire was removed.

5. The microcatheter was pre-filled with PG/PBS solution to aid in test article delivery.

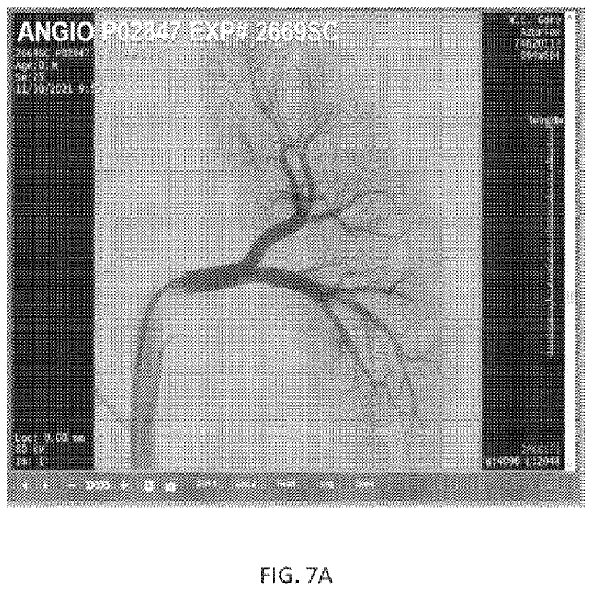

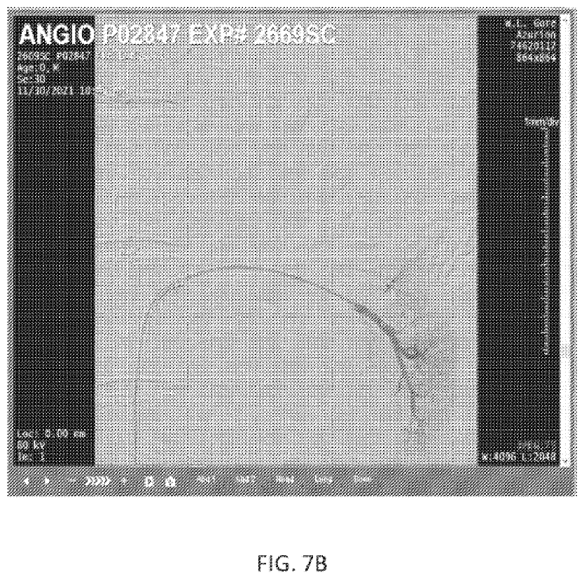

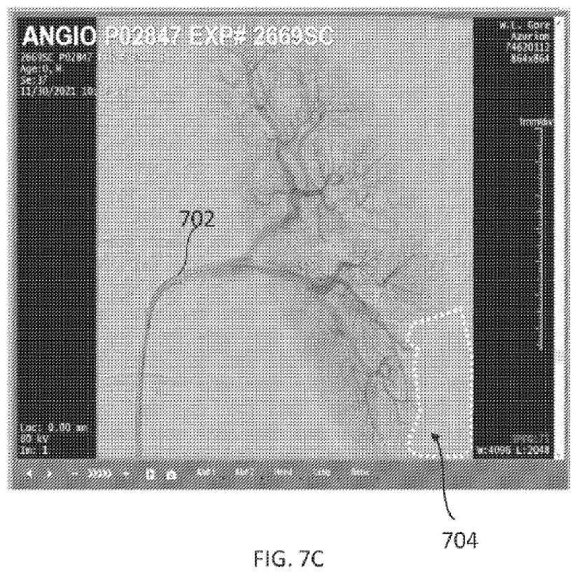

6. The test article was administered under fluoroscopic guidance. A first fluoroscopy image was taken before embolization, as shown in FIG. 7A.

7. Serial angiograms (i.e., fluoroscopy images) were used by injecting contrast through the guide-catheter to assess successful embolization/occlusion of the target artery. As shown in FIG. 7B, contrast was delivered through microcatheter 702 to show where the embolization would take place before delivery of the dispersion from Example 1. FIG. 7C includes another fluoroscopy image which was taken after embolization was complete. As shown in FIG. 7C, the vasculature in area 704 no longer has blood flow, thus indicating successful embolization in area 704.

8. Additional embolic material may be injected as needed to achieve complete occlusion of the target site.

9. The microcatheter was then removed.

10. A final angiogram may be performed to ensure the success of treatment.

### Example 21: Deployment of Composition in a Venous Sheep Model

A dispersion of TFE-VOH copolymer particles uniformly distributed throughout a PG/PBS solution (liquid carrier) was prepared using the method described above with the composition of 1% and 3% TFE-VOH 60/40 PG/PBS. It was demonstrated that the composition injected into a vein via a syringe will displace the blood within that vein with the composition remaining in the vein occluding the vein. As the composition is injected into the vein, the composition is advanced downstream as the composition is dispensed from the hypodermic needle tip. This demonstration exemplifies using the composition to treat varicose or spider veins effectively displacing the blood and replaced by the composition.

In accordance with an embodiment, the viscosity of the composition may be predetermined by choosing a predetermined weight percent of TFE-VOH to carrier liquid. By way of example, the viscosity of the composition increases when the weight percent of TFE-VOH to carrier liquid increases, such as, by way of example, the viscosity of 0.5 wt % TFE-VOH to carrier liquid is less than the viscosity of 1.0 wt % TFE-VOH to carrier liquid which has a lower viscosity than 3.5 wt % TFE-VOH to carrier liquid, which has a lower viscosity than 5.0 wt % TFE-VOH to carrier liquid.

In accordance with an embodiment, the polymer assembly rate may be predetermined by choosing a predetermined ratio of PG to carrier liquid, such as PG/Saline and PG/PBS. By way of example, some embodiments herein have a ratio of PG/PBS of 60/40. The polymer assembly rate has been determined to increase as the ratio of PG/PBS increases, such that a 65/35 PG/Saline ratio is slower to self assemble vs 50/50 PG/Saline ratio which will increase the assembly rate. In other words, the greater the amount of PG to carrier liquid, the slower the polymer assembly rate.

Numerous characteristics and advantages of the present disclosure have been set forth in the preceding description, including preferred and alternate embodiments together with details of the structure and function of the disclosure. The disclosure is intended as illustrative only and as such is not intended to be exhaustive. It will be evident to those skilled in the art that various modifications can be made, especially in matters of structure, materials, elements, components, shape, size and arrangement of parts within the principals of the disclosure, to the full extent indicated by the broad, general meaning of the terms in which the appended claims are expressed. To the extent that these various modifications do not depart from the spirit and scope of the appended claims, they are intended to be encompassed therein. In addition to being directed to the embodiments described above and claimed below, the present disclosure is further directed to embodiments having different combinations of the features described above and claimed below. As such, the disclosure is also directed to other embodiments having any other possible combination of the dependent features claimed below.

What is claimed is:

1. A composition comprising:
a tetrafluoroethylene copolymer dispersed in a liquid carrier;
wherein a first ratio of a weight of the tetrafluoroethylene copolymer to a volume of the liquid carrier is from about 0.01 g/ml to about 0.1 g/ml.

2. The composition of claim 1, wherein the first ratio is from about 0.02 g/ml to about 0.08 g/ml.

3. The composition of claim 2, wherein the first ratio is from about 0.03 g/ml to about 0.06 g/ml.

4. The composition of any one of claims 1-3, further comprising:
a radiopacity agent suspended in the liquid carrier;
wherein a second ratio of a weight of the radiopacity agent to the volume of the liquid carrier is from about 0.1 g/ml to about 0.75 g/ml.

5. The composition of claim 4, wherein the second ratio is from about 0.12 g/ml to about 0.5 g/ml.

6. The composition of claim 5, wherein the second ratio is from about 0.3 g/ml to about 0.4 g/ml.

7. The composition of any of the preceding claims, wherein the liquid carrier comprises a water miscible organic component and an aqueous component;
wherein the ratio of the water miscible organic component to the aqueous component is from about 1:1 to about 4:1.

8. The composition of claim 7, wherein the ratio of the water miscible organic component to the aqueous component is from about 3:2 to about 7:3.

9. The composition of claim 7, wherein the water miscible organic component is selected from a group consisting of alcohols, esters, ketones, glycols, and aldehydes, and polar aprotic solvents.

10. The composition of claim 7, wherein the water miscible organic component is selected from the group consisting of FDA Class III Solvents.

11. The composition of claim 7, wherein the water miscible organic component comprises propylene glycol (PG), ethanol, or dimethyl sulfoxide (DMSO).

12. The composition of claim 7, wherein the water miscible organic component is configured to stabilize the tetrafluoroethylene copolymer dispersed in the liquid carrier by forming physical crosslinks with the tetrafluoroethylene copolymer.

13. The composition of claim 12, wherein the physical crosslinks comprise hydrogen bonds.

14. The composition of claim 7, wherein the aqueous component comprises water or saline comprising phosphate buffered saline (PBS) or saline.

15. The composition of any of claims 4 to 14, wherein the composition is visible within a patient's body under fluoroscopy with wavelength of from about 0.01 to about 10 nm.

16. The composition of any one of claims 4 to 15, wherein the radiopacity agent is selected from a group consisting of tantalum, barium sulfate, iohexol, iodixanol, ioxaglic acid, lipiodol, and triiodobenzoic acid.

17. The composition of any of the preceding claims, wherein the tetrafluoroethylene copolymer comprises a hydroxyl functional group.

18. The composition of any of the preceding claims, wherein the tetrafluoroethylene copolymer is poly(tetrafluoroethylene-co-vinyl alcohol) (TFE-VOH).

19. The composition of any of the preceding claims, wherein the tetrafluoroethylene copolymer has a molecular weight of from about 50,000 g/mol to about 500,000 g/mol.

20. The composition of any of the preceding claims, wherein the tetrafluoroethylene copolymer has a molecular size of from about 33 nm to about 165 nm.

21. The composition of any of the preceding claims, wherein the composition is configured to be injected into a target space to form a gel that occludes at least a portion of the space.

22. The composition of claim 21, wherein the target space is within a vessel, an organ, or a tissue of a patient.

23. The composition of claim 21 or 22, wherein the composition is injected using a delivery device including a needle, a catheter, a microcatheter, or a syringe.

24. The composition of any one of claims 21 to 23, wherein the target space is adjacent to a distal end of the delivery device.

25. The composition of any one of claims 21 to 23, wherein the target space is downstream and a distance away from the distal end of the delivery device, wherein the composition is conveyed to the target site by the fluid flowing through the vessel.

26. The composition of any one of claims 21 to 25, wherein the composition is an embolic agent used for an embolization procedure.

27. The composition of any one of claims 21 to 26, wherein the composition is stirred or agitated before injection.

28. The composition of any one of claims 21 to 27, wherein the tetrafluoroethylene copolymer and the radiopacity agent are evenly distributed within the gel formed at the target space.

29. The composition of claim 1, wherein the tetrafluoroethylene copolymer dispersed in the liquid carrier forms a dispersion kinetically stable at 25° C. for at least 5 minutes without stirring or agitation.

30. The composition of claim 29, wherein the dispersion is kinetically stable at 25° C. for at least 4 months without stirring or agitation.

31. The composition of claim 29, wherein the dispersion is kinetically stable at 25° C. for at least 2 years without stirring or agitation.

32. A method of preparing a composition comprising:
mixing a tetrafluoroethylene copolymer with a water miscible organic component to form a mixture;
mixing an aqueous component with the mixture to form a kinetically stable dispersion; and
wherein the composition comprises the tetrafluoroethylene copolymer uniformly distributed throughout a liquid carrier comprising the water miscible organic component and the aqueous component.

33. The method of claim 32, further comprising adding a radiopacity agent to the kinetically stable dispersion to form the composition, and wherein the radiopacity agent is suspended in the liquid carrier.

34. A method of preparing a composition comprising:
mixing a tetrafluoroethylene copolymer with a liquid carrier comprising a water miscible organic component and an aqueous component to form a kinetically stable dispersion; and
wherein the composition comprises the tetrafluoroethylene copolymer uniformly distributed throughout a liquid carrier comprising the water miscible organic component and the aqueous component.

35. The method of claim 32, further comprising adding a radiopacity agent to the dispersion to form the composition, and wherein the radiopacity agent is suspended in the liquid carrier.

36. The method of claim 32 or 34, wherein the mixing steps are conducted using a tumbling fixture at a temperature of from about 65° C. to about 105° C.

37. The method of claim 32 or 34, wherein the mixing steps are conducted using a shaker plate at a temperature of from about 60° C. to about 100° C.

38. The method of claim 32 or 34, wherein the mixing steps are conducted via stirring at a temperature of from about 60° C. to about 100° C.

39. The method of any one of claims 32 to 38, wherein the radiopacity comprises tantalum.

40. A method of preparing a composition comprising:
mixing a tetrafluoroethylene copolymer with a water miscible organic component to form a mixture;
mixing an aqueous component with the mixture to form a kinetically stable dispersion; and
wherein the composition comprises the tetrafluoroethylene copolymer uniformly distributed throughout a liquid carrier comprising the water miscible organic component and the aqueous component.

41. The method of claim 40, further comprising mixing a radiopacity agent with the kinetically stable dispersion to form the composition, and wherein the radiopacity agent is uniformly distributed throughout a liquid carrier including the water miscible organic component and the aqueous component.

42. The method of claim 40 or 41, wherein the first and second mixing steps are conducted using a tumbling fixture at a temperature of from about 65° C. to about 105° C.

43. The method of claim 40 or 41, wherein the first and second mixing steps are conducted using a shaker plate at a temperature of from about 60° C. to about 100° C.

44. The method of claim 40 or 41, wherein the first and second mixing steps are conducted via stirring at a temperature of from about 60° C. to about 100° C.

45. The method of any one of claims 40 to 44, wherein the third mixing step is conducted at a temperature of from about 30° C. to about 70° C.

46. A method of preparing a composition comprising:
mixing a tetrafluoroethylene copolymer with a liquid carrier comprising a water miscible organic component and an aqueous component to form a kinetically stable dispersion; and
wherein the composition comprises the tetrafluoroethylene copolymer uniformly distributed throughout a liquid carrier comprising the water miscible organic component and the aqueous component.

47. The method of claim 46, further comprising adding a radiopacity agent to the kinetically stable dispersion to form the composition, and wherein the radiopacity agent is suspended in the liquid carrier.

48. The method of claim 46 or 47, wherein the first mixing step is conducted using a tumbling fixture at a temperature of from about 65° C. to about 105° C.

49. The method of claim 46 or 47, wherein the first mixing step is conducted using a shaker plate at a temperature of from about 60° C. to about 100° C.

50. The method of claim 46 or 47, wherein the first mixing step is conducted via stirring at a temperature of from about 60° C. to about 100° C.

51. The method of any one of claims 46 to 50, wherein the second mixing step is conducted at a temperature of from about 30° C. to about 70° C.

52. The method of any one of claims 37 to 46, wherein the radiopacity agent comprises iohexol.

53. A method comprising:
providing a composition comprising a tetrafluoroethylene copolymer uniformly distributed throughout a liquid carrier, the liquid carrier comprising a water miscible organic component and an aqueous component;
introducing the composition into a patient at a target space; and
diffusing the water miscible organic component into the patient to form a gel comprising the tetrafluoroethylene copolymer at the target space.

54. The method of claim 53, further comprising a radiopacity agent suspended in the liquid carrier.

55. The method of claim 53 or 54, wherein the composition is introduced into an occlusion site or a bulking site of the patient.

56. The method of claim 55, wherein the occlusion site or the bulking site is within a vessel, an organ, or a tissue of the patient.

57. The method of any one of claims 53 to 56, wherein the introducing step is performed using a delivery device including a needle, a catheter, a microcatheter, or a syringe.

58. The method of any one of claims 53 to 57, wherein the gel is substantially porous.

59. The method of any one of claims 53 to 57, wherein the gel is permeable.

60. The method of any one of claims 53 to 59, wherein the composition is an embolic agent used for an embolization procedure.

61. The method of any one of claims 53 to 60,
wherein the radiopacity agent comprises tantalum;
wherein the composition is stirred or agitated before the introducing step.

62. The composition of any one of claims 53 to 61, wherein the tetrafluoroethylene copolymer and the radiopacity agent are evenly distributed within the gel formed at the target space.

63. A method of treating disease by providing distal embolization comprising providing a composition including a tetrafluoroethylene copolymer uniformly distributed throughout a liquid carrier, the liquid carrier including a water miscible organic component and an aqueous component; and deploying the composition through a delivery system operable to deliver the composition to a target site.

64. The method of claim 63, wherein deploying the composition through a delivery system comprises preparing a microcatheter by priming with a primer solution; and deploying the composition through the microcatheter operable to deliver the composition to a target site in peripheral arterial vasculature; and achieving occlusion of the target site.

65. The method of claim 63, wherein deploying the composition through the microcatheter operable to deliver the composition to a target site further comprises deploying the composition through the microcatheter operable to deliver the composition to a target site adjacent to the microcatheter distal end.

66. The method of claim 63, wherein deploying the composition through the microcatheter operable to deliver the composition to a target site further comprises deploying the composition through the microcatheter operable to deliver the composition to a target site downstream and a distance way from the microcatheter distal end, wherein the composition is conveyed by the flow of blood through the vessel.

67. The method of claim 63, wherein deploying the composition further comprises deploying the composition through the microcatheter operable to deliver the composition to a target site so as to achieve occlusion of the target site.

68. The method of claim 63, wherein deploying the composition further comprises deploying the composition through the microcatheter operable to deliver the composition to a target site so as to achieve occlusion of the target site in peripheral arterial vasculature.

69. The method of any one of claims 63 to 68, wherein the distal embolization is for treatment of hemorrhage.

70. The method of any one of claims 63 to 68, wherein the distal embolization is for treatment of hypervascular tumors.

71. The method of any one of claims 63 to 68, wherein the distal embolization is for treatment of pseudoaneurysms.

72. The method of any one of claims 63 to 68, wherein the distal embolization is for treatment of aneurysms.

73. The method of any one of claims 63 to 68, wherein the distal embolization is for renal embolization.

74. The method of any one of claims 63 to 68, wherein the distal embolization is for neurovascular indications.

75. The method of any one of claims 69 to 73, wherein the distal embolization is performed within or involves the portal vein.
